SEQUENCE LISTING (6 sequences)

SEQ ID NO: 1 - DNA, 28 bp, Artificial Sequence
  Synthetic nucleotide
aattggcgcgcccgggactagtcttggg

SEQ ID NO: 2 - DNA, 28 bp, Artificial Sequence
  Synthetic nucleotide
aattcccaagactagtcccgggcgcgcc

SEQ ID NO: 3 - DNA, 30 bp, Artificial Sequence
  Synthetic nucleotide
aaaggcgcgccgcgaaggtaaagtcagccg

SEQ ID NO: 4 - DNA, 30 bp, Artificial Sequence
  Synthetic nucleotide
aaaactagtcaggagtgaattaagtcattg

SEQ ID NO: 5 - DNA, 28 bp, Artificial Sequence
  Synthetic nucleotide
tgaatgaatgaactagttcaatcactca

SEQ ID NO: 6 - DNA, 28 bp, Artificial Sequence
  Synthetic nucleotide
tgagtgattgaactagttcattcattca

(19) United States
(12) Patent Application Publication
(10) Pub. No.: US 2015/0238596 A1
(43) Pub. Date: Aug. 27, 2015

(54) BIOCONJUGATES COMPRISING MODIFIED ANTIGENS AND USES THEREOF

(71) Applicant: GLYCOVAXYN AG, Schlieren (CH)

(72) Inventors: Michael Wacker, Unterengstringen (CH); Michael Kowarik, Zurich (CH); Michael Wetter, Zurich (CH)

(73) Assignee: GlycoVaxyn AG, Schlieren (CH)

(21) Appl. No.: 14/426,496

(22) PCT Filed: Sep. 10, 2013
(86) PCT No.: PCT/EP2013/068737

§ 371 Date: Mar. 6, 2015

Related U.S. Application Data
(60) Provisional application No. 61/698,843, filed on Sep. 10, 2012.

(51) Int. Cl. A61K 39/385; A61K 39/108; A61K 47/48; C12N 9/10

(52) U.S. Cl. CPC A61K 39/385; C12N 9/1081; A61K 39/0258; A61K 47/48261; A61K 47/4833; A61K 2039/6037

(57) ABSTRACT

Provided herein is a bioconjugate comprising a carrier protein and a modified antigen of Escherichia coli, the O antigen 0121. Also provided herein are uses of said bioconjugate, such as the treatment and/or prevention of diseases caused by Salmonella enterica, including diseases caused by Salmonella enterica subspecies I serovar Typhi (S. typhi).

This application claims priority to U.S. provisional patent application No. 61/698,843 filed Sep. 10, 2012, the disclosure of which is herein incorporated by reference in its entirety.

### 1. INTRODUCTION

Provided herein is a modified antigen of Escherichia coli, the O antigen from E. coli serovar O121. Also provided herein are uses of the modified E. coli O121 O-antigen, such as use of the modified E. coli O121 O-antigen in the treatment and/or prevention of disease, e.g., treatment and/or prevention of disease caused by Salmonella enterica, including disease caused by Salmonella enterica subspecies I serovar Typhi (S. typhi).

### 2. BACKGROUND

Typhoid fever remains a serious public health problem of which there are 22-33 million cases occurring each year, including about 216'000-500'000 deaths [Crump, J. A., Luby, S. P., Mintz, E. D.: The global burden of typhoid fever. Bull World Health Organ 82(5), 346-353 (2004)]. The causative agent of this human systemic infection, Salmonella enterica subspecies I serovar typhi (S. typhi), is feco-orally transmitted through contaminated water and food. Hence, typhoid fever is endemic in less developed areas where sanitary conditions remain poor. This includes many countries of Asia, Africa and South America, where schoolchildren and young adults are most frequently affected [Bhan, M. K., Bahl, R., Bhatnagar, S.: Typhoid and paratyphoid fever. Lancet 366(9487), 749-762 (2005)]. Antimicrobial treatment of typhoid fever has become increasingly complicated through the emergence of multidrug resistant strains of S. typhi [Mirza, S. H., Beeching, N. J., Hart, C. A.: Multi-drug resistant typhoid: a global problem. J Med Microbiol 44(5), 317-319 (1996)].

Vaccination of high-risk populations is considered the most promising strategy for the control and prevention of typhoid fever. Currently, there are two licensed typhoid vaccines: the orally administered, live attenuated whole cell vaccine Ty21a and the purified Vi polysaccharide parenteral vaccine. The Ty21a vaccine has several disadvantages: (i) the mutations contributing to the attenuated phenotype of this S. typhi strain are not fully defined [Hone, D. M., Attridge, S. R., Forrest, B., Morona, R., Daniels, D., LaBrooy, J. T., Bartholomeusz, R. C., Shearman, D. J., Hackett, J.: A galE via (Vi antigen-negative) mutant of Salmonella typhi Ty2 retains virulence in humans. Infect Immun 56(5), 1326-1333 (1988)], (ii) attenuated strains could theoretically revert to virulence, and (iii) Ty21a is only modestly immunogenic and requires three to four initial doses and boosters every 5 years [Levine, M. M., Ferreccio, C., Black, R. E., Germanier, R.: Large-scale field trial of Ty21a live oral typhoid vaccine in enteric-coated capsule formulation. Lancet 1(8541), 1049-1052 (1987); Black, R. E., Levine, M. M., Ferreccio, C., Clements, M. L., Lanata, C., Rooney, J., Germanier, R.: Efficacy of one or two doses of Ty21a Salmonella typhi vaccine in enteric-coated capsules in a controlled field trial. Chilean Typhoid Committee. Vaccine 8(1), 81-84 (1990); Murphy, J. R., Grez, L., Schlesinger, L., Ferreccio, C., Baqar, S., Munoz, C., Wasserman, S. S., Losonsky, G., Olson, J. G., Levine, M. M.: Immunogenicity of Salmonella typhi Ty21a vaccine for young children. Infect Immun 59(11), 4291-4293 (1991); Levine, M. M., Ferreccio, C., Cryz, S., Ortiz, E.: Comparison of enteric-coated capsules and liquid formulation of Ty21a typhoid vaccine in randomised controlled field trial. Lancet 336(8720), 891-894 (1990); Levine, M. M., Ferreccio, C., Abrego, P., Martin, O. S., Ortiz, E., Cryz, S.: Duration of efficacy of Ty21a, attenuated Salmonella typhi live oral vaccine. Vaccine 17 Suppl 2, S22-27 (1999)]. The usefulness of the Vi polysaccharide vaccine is limited by its age-related immunogenicity and the fact that immune responses against polysaccharides are T cell independent. Therefore, immunological memory cannot be established and revaccination does not elicit any booster response [Weintraub, A.: Immunology of bacterial polysaccharide antigens. Carbohydr Res 338(23), 2539-2547 (2003), Landy, M.: Studies on Vi antigen. VI. Immunization of human beings with purified Vi antigen. Am J Hyg 60(1), 52-62 (1954)]. Due to these drawbacks, the replacement of current typhoid vaccines with well defined, well tolerated and highly immunogenic vaccines is desirable.

The disadvantages of a polysaccharide vaccine can be overcome by conjugating the carbohydrate to a protein carrier (conjugate vaccine). Upon conjugation, the polysaccharide behaves like a T cell dependent antigen. It has been shown that purified Vi polysaccharide covalently linked to recombinant Pseudomonas aeruginosa exotoxin A (EPA) induces a protective immune response against S. typhi in young children [Szu, S. C., Taylor, D. N., Trofa, A. C., Clements, J. D., Shiloach, J., Sadoff, J. C., Bryla, D. A., Robbins, J. B.: Laboratory and preliminary clinical characterization of Vi capsular polysaccharide-protein conjugate vaccines. Infect Immun 62(10), 4440-4444 (1994); Lin, F. Y., Ho, V. A., Khiem, H. B., Trach, D. D., Bay, P. V., Thanh, T. C., Kossaczka, Z., Bryla, D. A., Shiloach, J., Robbins, J. B., Schneerson, R., Szu, S. C.: The efficacy of a Salmonella typhi Vi conjugate vaccine in two-to-five-year-old children. N Engl J Med 344(17), 1263-1269 (2001)]. However, production of conjugate vaccines is a complex, multi-step process. First, separate bacterial strains producing the recombinant protein carrier and the polysaccharide antigen have to be cultivated. The polysaccharide and the protein carrier have to be purified by different procedures, before the two components are chemically coupled. The last step involves additional purification steps for obtaining the final product. This laborious production process has disadvantages: (i) several purification steps are required, where considerable losses might occur and (ii) due to the random nature of chemical coupling the product is not a uniform structure but a mixture of different glycoconjugates, with potentially different efficacy profiles.

Thus, there remains a need for improved methods of treating and preventing infection of subjects with Salmonella enterica, including infection with S. typhi.

### 3. SUMMARY

In one aspect, provided herein are bioconjugates comprising a carrier protein and a modified E. coli O121 O-antigen.

In certain embodiments, the modified E. coli O121 O-antigen of the bioconjugates provided herein is covalently bound to an asparagine residue (Asn) within a glycosylation site of the carrier protein, wherein the glycosylation site comprises the amino acid sequence Asp/Glu-X-Asn-Z-Ser/Thr wherein X and Z may be any amino acid except Pro. In certain embodiments, the carrier proteins of the bioconjugates provided herein do not naturally (e.g., in their normal/native, or “wild-type” state) comprise a glycosylation site. In certain embodiments, the carrier proteins of the bioconjugates provided herein are engineered to comprise one or more glycosylation sites, e.g., the carrier proteins are engineered to comprise one or more glycosylation sites comprising the amino acid sequence Asp/Glu-X-Asn-Z-Ser/Thr wherein X and Z may be any amino acid except Pro. For example, the carrier proteins used in accordance with the methods described herein may comprise 1, 2, 3, 4, 5, 6, 7, 8, 9, 10 or more glycosylation sites, each having the amino acid sequence Asp/Glu-X-Asn-Z-Ser/Thr, wherein X and Z may be any amino acid except Pro; and wherein some (e.g., 1, 2, 3, 4, 5, 6, 7, 8, or 9) or all of the glycosylation sites have been recombinantly introduced into the carrier protein.

Any carrier proteins suitable for use in the methods described herein (e.g., treatment and/or prevention of S. typhi infection) can be used in the generation of the bioconjugates described herein. Exemplary carrier proteins include, without limitation, Exotoxin A of P. aeruginosa (EPA), CRM 197, Diphtheria toxoid, tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit (CTB), cholera toxin, detoxified variants of cholera toxin, E. coli sat protein, the passenger domain of E. coli sat protein, C. jejuni AcrA, and C. jejuni natural glycoproteins.

In a specific embodiment, provided herein is a bioconjugate comprising a carrier protein and a modified E. coli O121 O-antigen, wherein the modified E. coli O121 O-antigen comprises:

→4)-α-D-GalNAcA-(1→4)-α-D-GalNAcA-(1→.

In another specific embodiment, provided herein is a bioconjugate comprising a carrier protein and a modified E. coli O121 O-antigen, wherein the modified E. coli O121 O-antigen comprises:

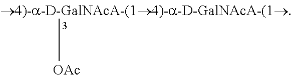

In another specific embodiment, provided herein is a bioconjugate comprising a carrier protein and a modified E. coli O121 O-antigen, wherein the modified E. coli O121 O-antigen comprises:

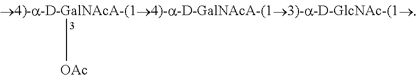

In another specific embodiment, provided herein is a bioconjugate comprising a carrier protein and a modified E. coli O121 O-antigen, wherein the modified E. coli O121 O-antigen comprises:

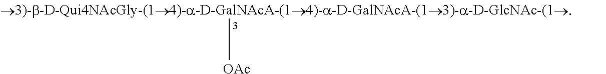

In another aspect, provided herein are prokaryotic host cells capable of producing the bioconjugates described herein. In a specific embodiment, provided herein is a prokaryotic host cell useful for generating a bioconjugate, wherein the prokaryotic host cell comprises: (i) a heterologous nucleotide sequence encoding a carrier protein comprising at least one glycosylation site comprising the amino acid sequence Asp/Glu-X-Asn-Z-Ser/Thr wherein X and Z may be any natural amino acid except Pro; and (ii) a heterologous nucleotide sequence encoding an oligosaccaryltransferase; wherein the prokaryotic host cell is recombinantly engineered to produce Und-PP-modified E. coli O121 O-antigen (i.e., any of the modified E. coli O121 O-antigens described herein), wherein the oligosaccharyl transferase transfers the modified E. coli O121 O-antigen to the Asn of the glycosylation site. In a specific embodiment, the prokaryotic host cells described herein are E. coli host cells. In another specific embodiment, the prokaryotic host cells described herein are E. coli strain K12 host cells. In another specific embodiment, the oligosaccharyl transferase recombinantly introduced into the host cells described herein, e.g., E. coli host cells, is PglB of Campylobacter jejuni.

In certain embodiments, the host cells described herein comprise heterologous nucleic acid sequences (i.e., nucleic acid sequences, e.g., genes, that are not normally associated with the host cell in its natural/native state, e.g., its “wild-type” state) in addition to heterologous oligosaccharyl transferases. Such additional heterologous nucleic acid sequences may comprise nucleic acids that encode genes that are known to be belong to glycosylation operons, e.g., prokaryotic glycosylation operons. In specific embodiments, such additional heterologous nucleic acid sequences comprise genes belonging to the pgl cluster of Campylobacter jejuni, or comprise the entire pgl cluster of Campylobacter jejuni.

In certain embodiments, the host cells described herein comprise one or more gene deletions and/or one or more gene inactivations, i.e., the genetic background of the host cells have been modified in such a way as to render one or more of the genes normally associated with the host cell (e.g., one or more “wild-type” genes) inactive or dysfunctional, or to remove the gene entirely. In a specific embodiment, the host cells used in the generation of the bioconjugates described herein are E. coli host cells, wherein said E. coli host cells have a mutation in, or deletion of, the wbqG gene. In another specific embodiment, the host cells used in the generation of the bioconjugates described herein are E. coli host cells, wherein said E. coli host cells have a mutation in, or deletion of, the wbqC gene. In another specific embodiment, the host cells used in the generation of the bioconjugates described herein are E. coli host cells, wherein said E. coli host cells have a mutation in, or deletion of, the wbqE gene. In another specific embodiment, the host cells used in the generation of the bioconjugates described herein are E. coli host cells, wherein said E. coli host cells have a mutation in, or deletion of, the wbqG gene and the wbqC gene. In another specific embodiment, the host cells used in the generation of the bioconjugates described herein are E. coli host cells, wherein said E. coli host cells have a mutation in, or deletion of, the wbqG gene and the wbqE gene. In another specific embodiment, the host cells used in the generation of the bioconjugates described herein are E. coli host cells, wherein said E. coli host cells have a mutation in, or deletion of, the wbqG gene, the wbqC gene, and the wbqE gene.

In certain embodiments, an O121 gene cluster of E. coli (e.g., the O121 gene cluster of E. coli O121 reference strain CCUG 11422; the O121 gene cluster described in Fratamico et al., 2003, J. Clin. Microbiol. 41(7):3379-3383) is introduced (e.g., recombinantly introduced) into the host cells described herein. In certain embodiments, the O121 gene cluster is introduced into a host cell that does not produce any O antigen, e.g., the host cell has been modified in a manner such that it does not produce any O antigen. In certain embodiments, one or more genes of the O121 gene cluster are functionally inactivated (e.g., deleted, mutated in a manner that inactivates the gene, etc.). In a specific embodiment, the wbqG gene of the O121 gene cluster introduced into the host cells described herein is functionally inactivated (e.g., deleted). In another specific embodiment, the wbqG gene and/or the wbqE gene of the O121 gene cluster introduced into the host cells described herein is functionally inactivated (e.g., deleted). In specific embodiments, such host cells are used to produce modified E. coli O121 O-antigens (i.e., any of the modified E. coli O121 O-antigens described herein).

In certain embodiments, in addition to or instead of one or more of the gene deletions described above, the host cells used in the generation of the bioconjugates described herein comprise a deletion/inactivation of one or more of the following genes: waaL (see, e.g., Feldman et al., 2005, PNAS USA 102:3016-3021), lipid A core biosynthesis cluster, galactose cluster, arabinose cluster, colonic acid cluster, capsular polysaccharide cluster, undecaprenol-p biosynthesis genes, und-P recycling genes, metabolic enzymes involved in nucleotide activated sugar biosynthesis, enterobacterial common antigen cluster, and prophage O antigen modification clusters like the gtrABS cluster.

In another aspect, provided herein are methods of generating the bioconjugates provided herein. In certain embodiments, the methods for generating the bioconjugates provided herein comprise culturing a host cell described herein under conditions suitable for the production of proteins, and isolating the bioconjugate. Those of skill in the art will recognize conditions suitable for the maintenance of growth of host cells such that the bioconjugates described herein can be produced by the host cells and subsequently isolated. Such methods are additionally encompassed by the working Examples provided herein (see Section 6).

In yet another aspect, provided herein are compositions, e.g., immunogenic compositions, comprising the bioconjugates described herein. In certain embodiments, the immunogenic compositions described herein comprise a bioconjugate described herein and one or more additional components, e.g., an adjuvant.

In a further aspect, provided herein are methods of treating or preventing an infection with Salmonella enterica, comprising administering to a subject infected with Salmonella enterica, or at risk of being infected with Salmonella enterica, a bioconjugate described herein, or a composition (e.g., an immunogenic composition) thereof. In specific embodiments, the Salmonella enterica is Salmonella enterica subspecies I serovar typhi (S. typhi).

### 3.1 Conventions and Abbreviations

E. coli O121 Escherichia coli serotype O121

PglB bacterial oligosaccharyl transferase PglB

Und-PP undecaprenyl pyrophosphate

ELISA enzyme-linked immunosorbent assay

EPA Pseudomonas aeruginosa exotoxin A

Vi capsular polysaccharide linear, acidic homopolymer of α-1,4-linked N-acetylgalactosaminuronic acid (D-GalNAcA) residues

viaB Vi biosynthetic gene cluster

ABC transporter ATP Binding Cassette

wzy polymerase gene

E. coli O121 wbqG E. coli O121 wbqG O antigen mutant containing 2-acetamido-2-deoxy-d-galacturonate (d-GalNAcA), instead of d-GalNAcAN

CPS capsular polysaccharide

D-GalNAcAN N-acetylgalactosaminuronamide

Residue a (1→3)-α-D-GlcNAc

Residue b (1→4)-α-D-GalNAcA

Residue c (1→4)-α-D-GalNAcAN(60% O-acetylated at C-3)

Residue c′ (1→4)-α-D-GalNAcA(30-40% O-acetylated at C-3)

Residue d (1→3)-β-D-Qui4NAcGly

LPS lipopolysaccharide

2AB 2-aminobenzamide

MS mass spectrometry

m/z mass-to-charge ratio

(CID) MS-MS collisionally induced dissociation mass spectrometry-mass spectrometry

Da Dalton, unit of mass

kDa KiloDalton

waaL O antigen ligase gene

Und-PP undecaprenyl pyrophosphate

Dol-PP dolichyl pyrophosphate

wzz O antigen chain length regulator gene

ECA enterobacterial common antigen

CWP cell wall polysaccharide

ELISA enzyme-linked immunosorbent assay

A₆₀₀ Optical Density at 600 nm

MES 2-(N-morpholino)ethanesulfonic acid, used in MES running buffer

TBAP tetrabutylammonium phosphate

TFA trifluoroacetic acid

PEG Polyethylene glycol

CHCA α-cyano-4-hydroxycinnamic acid

IPTG isopropyl β-D-1-thiogalactopyranoside

CTAB hexadecyltrimethylammonium bromide

rpm Revolution per

BCA bicinchoninic acid assay

Vi-Tyr Tyraminated Vi polysaccharide

HRP horseradish peroxidase

TMB 3,3′, 5,5′-tetramethybenzidine

### 4. DESCRIPTION OF THE FIGURES

FIG. 1: Structure of the Salmonella typhi Vi polysaccharide and the repeating unit of the Escherichia coli O121 O antigen. Mutation of the O121 O antigen cluster encoded gene wbqG results in expression of a modified O polysaccharide structure. GalNAcA: 2-acetamido-2-deoxy-D-galacturonic acid; GalNAcAN: 2-acetamido-2-deoxy-D-galacturonamide; Qui4N: 4-amino-4,6-dideoxy-D-glucose.

FIG. 2: O polysaccharide analysis of E. coli O121 and its wbqG mutant derivative. (A) LPS from E. coli W3110 cells expressing the O121 wild type O antigen gene cluster or its wbqG mutant derivative was separated by SDS-PAGE and stained with silver or after transfer to a nitrocellulose membrane detected with anti-O121 and anti-Vi antibodies. Mutation of wbqG results in the assembly of a modified O antigen reactive with anti-Vi sera. (B) Und-PP-linked glycans were extracted from E. coli SCM6 cells expressing the O121 wild type O antigen gene cluster or its wbqG mutant derivative followed by 2AB labeling and separation by normal phase HPLC using a GlycoSep N column. Individual peak fractions were analyzed by mass spectrometry and the identified glycan structures are indicated. ▪: N-acetylhexosamine; [symbol] : dideoxyhexosamine; ⋄: hexuronic acid; ⋄^{N}: hexuronamide; Ac: acetyl; NAc: N-acetyl.

FIG. 3: CID MS-MS spectra of glycan species separated by normal phase HPLC. The CID MS-MS spectra correspond to the glycan species identified in the individual peak fractions seen in FIG. 2B with the following retention times: (A) 58.8 min, (B) 65.1 min, (C) 67.2 min, and (D) 73.5 min.

FIG. 4: Production of glycoconjugates using the bacterial N-glycosylation system. Glycoconjugates were produced in E. coli CLM24 by co-expressing the bacterial oligosaccharyl transferase PglB, the engineered carrier protein EPA, and genes driving the synthesis of an antigenic polysaccharide (E. coli O121, E. coli O121 wbqG mutant, Shigella dysenteriae O1). Purified glycoconjugates were analyzed by SDS-PAGE, followed by Coomassie blue staining or by western blot after transfer to nitrocellulose membranes using anti-EPA, anti-O121, and anti-Vi antibodies.

FIG. 5: Immunization studies with glycoconjugates. Groups of mice were immunized with purified glycoconjugates in the presence of Aluminum hydroxide. The control group was immunized with purified Vi polysaccharide. (A) Anti-O121 total immunoglobulin titers of sera collected on day 67. (B) Anti-Vi antibody titers of sera collected on day 67. Data is represented as individual () and mean (−) titers. One animal immunized with the O121_{wbqG}-EPA conjugate did not develop an O121-LPS specific antibody response, but the same animal showed a significant rise in anti-Vi antibody titer.

### 5. DETAILED DESCRIPTION

In one aspect, provided herein are bioconjugates comprising a carrier protein and a modified E. coli O121 O-antigen.

In another aspect, provided herein are prokaryotic host cells capable of producing the bioconjugates described herein. In a specific embodiment, provided herein is a prokaryotic host cell useful for generating a bioconjugate, wherein the prokaryotic host cell comprises: (i) a heterologous nucleotide sequence encoding a carrier protein comprising at least one glycosylation site comprising the amino acid sequence Asp/Glu-X-Asn-Z-Ser/Thr wherein X and Z may be any natural amino acid except Pro; and (ii) a heterologous nucleotide sequence encoding an oligosaccharyl transferase; wherein the prokaryotic host cell is recombinantly engineered to produce Und-PP-modified E. coli O121 O-antigen (i.e., any of the modified E. coli O121 O-antigens described herein), wherein the oligosaccharyl transferase transfers the modified E. coli O121 O-antigen to the Asn of the glycosylation site.

In another aspect, provided herein are methods of generating the bioconjugates provided herein. In certain embodiments, the methods for generating the bioconjugates provided herein comprise culturing a host cell described herein under conditions suitable for the production of proteins, and isolating the bioconjugate.

In yet another aspect, provided herein are compositions, e.g., immunogenic compositions, comprising the bioconjugates described herein. In a further aspect, provided herein are methods of treating or preventing an infection with Salmonella enterica, comprising administering to a subject infected with Salmonella enterica, or at risk of being infected with Salmonella enterica, a bioconjugate described herein, or a composition (e.g., an immunogenic composition) thereof. In specific embodiments, the Salmonella enterica is Salmonella enterica subspecies I serovar typhi (S. typhi).

### 5.1 Host Cells

Any host cells can be used to produce the bioconjugates described herein. In specific embodiments, the host cells used to produce the bioconjugates described herein are prokaryotic host cells. Exemplary prokaryotic host cells include, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species., Bacillus species, and Clostridium species. In a specific embodiment, the host cells used i to produce the bioconjugates described herein are Escherichia coli (E. coli) host cells (e.g., E. coli strain K12 or CLM 24 and derivatives thereof).

In certain embodiments, the host cells used to produce the bioconjugates described herein are engineered to comprise heterologous nucleic acids, e.g., heterologous nucleic acids that encode one or more carrier proteins (see, e.g., Section 5.2) and/or heterologous nucleic acids that encode one or more proteins, e.g., genes encoding one or more proteins (see, e.g., Section 5.1.1). In a specific embodiment, heterologous nucleic acids that encode proteins involved in glycosylation pathways (e.g., prokaryotic and/or eukaryotic glycosylation pathways) may be introduced into the host cells described herein. Such nucleic acids may encode proteins including, without limitation, oligosaccharyl transferases and/or glycosyltransferases. Heterologous nucleic acids (e.g., nucleic acids that encode carrier proteins and/or nucleic acids that encode other proteins, e.g., proteins involved in glycosylation) can be introduced into the host cells described herein using any methods known to those of skill in the art, e.g., electroporation, chemical transformation by heat shock, natural transformation, phage transduction, and conjugation. In specific embodiments, heterologous nucleic acids are introduced into the host cells described herein using a plasmid, e.g., the heterologous nucleic acids are expressed in the host cells by a plasmid (e.g., an expression vector).

In certain embodiments, additional modifications may be introduced (e.g., using recombinant techniques) into the host cells described herein. For example, host cell nucleic acids (e.g., genes) that encode proteins that form part of a possibly competing or interfering glycosylation pathway (e.g., compete or interfere with one or more heterologous genes involved in glycosylation that are recombinantly introduced into the host cell) can be deleted or modified in the host cell background (genome) in a manner that makes them inactive/dysfunctional (i.e., the host cell nucleic acids that are deleted/modified do not encode a functional protein or do not encode a protein whatsoever). In certain embodiments, when nucleic acids are deleted from the genome of the host cells provided herein, they are replaced by a desirable sequence, e.g., a sequence that is useful for glycoprotein production. Exemplary genes that can be deleted in host cells (and, in some cases, replaced with other desired nucleic acid sequences) include genes of the host cells involved in glycolipid biosynthesis, such as waaL (see, e.g., Feldman et al., 2005, PNAS USA 102:3016-3021), lipid A core biosynthesis cluster, galactose cluster, arabinose cluster, colonic acid cluster, capsular polysaccharide cluster, undecaprenol-p biosynthesis genes, und-P recycling genes, metabolic enzymes involved in nucleotide activated sugar biosynthesis, enterobacterial common antigen cluster, and prophage O antigen modification clusters like the gtrABS cluster. In a specific embodiment, the host cells described herein are modified such that they do not produce any O antigens other than the modified E. coli O121 O-antigens described herein (e.g., the host cell machinery for producing O antigens other than the modified E. coli O121 O-antigens described herein is deleted/inactivated).

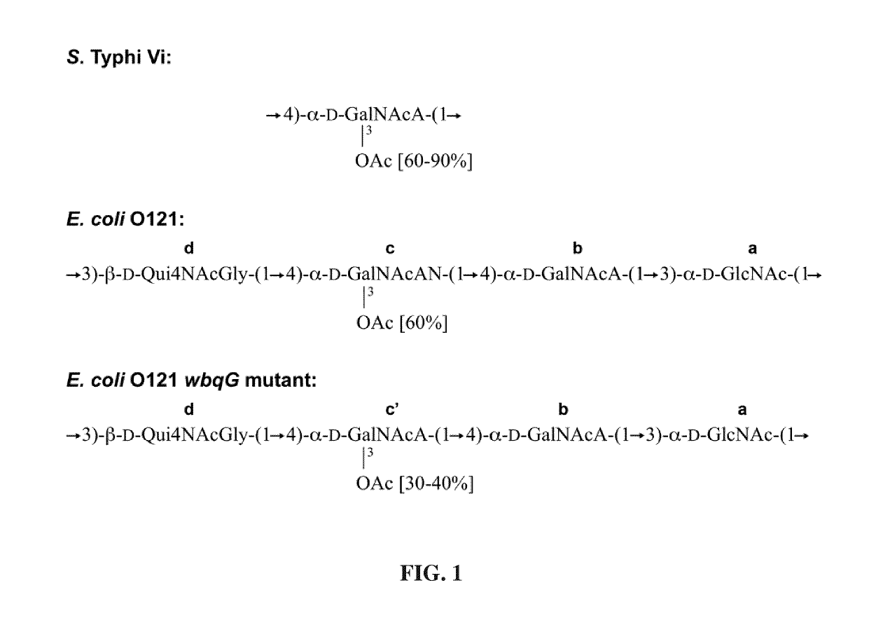

In specific embodiments, the genome of the host cells described herein can be modified in such a manner that one or more genes involved in the production of antigens that become associated with the bioconjugates described herein are no longer produced by the host cell. For example, one or more genes involved in the production of an antigenic side chain that would, under normal circumstances, be associated with the bioconjugates described herein can be deleted. Without intending to be bound by any particular theory of operation, it is believed that inactivation/deletion nucleic acids that encode genes involved in the production of antigens that become associated with the bioconjugates described herein, other than the modified E. coli O121 O-antigens described herein (e.g., antigenic side chains), increases/enhances the specific immune response directed against the modified E. coli O121 O-antigens described herein, thus increasing the antigenicity of the bioconjugates described herein. In a specific embodiment, the host cells described herein possess a mutated/deleted/inactivated wbqC gene, resulting in inactivation/deletion of the AcGly side chain (i.e., residue d in FIG. 1). In another specific embodiment, the host cells described herein possess a mutated/deleted/inactivated wbqE gene.

In a specific embodiment, the host cells used in the generation of the bioconjugates described herein are E. coli host cells, wherein said E. coli host cells have a mutation in, or deletion of, the wbqG gene. In another specific embodiment, the host cells used in the generation of the bioconjugates described herein are E. coli host cells, wherein said E. coli host cells have a mutation in, or deletion of, the wbqC gene. In another specific embodiment, the host cells used in the generation of the bioconjugates described herein are E. coli host cells, wherein said E. coli host cells have a mutation in, or deletion of, the wbqE gene. In another specific embodiment, the host cells used in the generation of the bioconjugates described herein are E. coli host cells, wherein said E. coli host cells have a mutation in, or deletion of, the wbqG gene and the wbqC gene. In another specific embodiment, the host cells used in the generation of the bioconjugates described herein are E. coli host cells, wherein said E. coli host cells have a mutation in, or deletion of, the wbqG gene and the wbqE gene. In another specific embodiment, the host cells used in the generation of the bioconjugates described herein are E. coli host cells, wherein said E. coli host cells have a mutation in, or deletion of, the wbqG gene, the wbqC gene, and the wbqE gene. Such host cells can further comprise any of the modifications described herein, e.g., the host cells comprise heterologous nucleic acids encoding a carrier protein and/or encoding one or more genes involved in protein glycosylation (e.g., an oligosaccharyl transferase) and/or the host cells may comprise further gene deletions/inactivations (e.g., deletion of waaL).

In certain embodiments, an O121 gene cluster of E. coli (e.g., the O121 gene cluster of E. coli O121 reference strain CCUG 11422; the O121 gene cluster described in Fratamico et al., 2003, J. Clin. Microbiol. 41(7):3379-3383) is introduced (e.g., recombinantly introduced) into the host cells described herein. In certain embodiments, the O121 gene cluster is introduced into a host cell that does not produce any O antigen, e.g., the host cell has been modified in a manner such that it does not produce any O antigen. In certain embodiments, one or more genes of the O121 gene cluster are functionally inactivated (e.g., deleted, mutated in a manner that inactivates the gene, etc.). In a specific embodiment, the wbqG gene of the O121 gene cluster introduced into the host cells described herein is functionally inactivated (e.g., deleted). In another specific embodiment, the wbqG gene and/or the wbqE gene of the O121 gene cluster introduced into the host cells described herein is functionally inactivated (e.g., deleted). In specific embodiments, such host cells are used to produce modified E. coli O121 O-antigens (i.e., any of the modified E. coli O121 O-antigens described herein).

### 5.1.1 Glycosylation Machinery

In certain embodiments, the host cells provided herein are modified to express glycosylation machinery such that the host cell is capable of producing a modified E. coli O121 O-antigen described herein. In even more specific embodiments, the glycosylation machinery of the host cell is engineered to produce a UndPP-linked modified E. coli O121 O-antigen.

Without being bound by theory, the UndPP-linked modified E. coli O121 O-antigen is then flipped from the cytosol of the host cell into the periplasmic space of the host cell. Further, without being bound by theory, the modified E. coli O121 O-antigen is then transferred from UndPP onto the carrier protein on an Asn of a glycosylation site of the carrier protein.

In certain embodiments, a heterologous nucleic acid encoding a glycosyltransferase is introduced (e.g., introduced using recombinant approaches) into the host cell so that a modified E. coli O121 O-antigen is generated on UndPP. Those of skill in the art will readily recognize that any suitable heterologous glycosyltransferases can be used in accordance with the methods described herein. In a specific embodiment a heterologous nucleic acid encoding a glycosyltransferase from C. jejuni is introduced into the host cell.

In certain embodiments, a heterologous nucleic acid encoding an oligosaccharyl transferase is introduced into the host cells described herein. Those of skill in the art will readily recognize that any suitable heterologous oligosaccharyl transferases can be used in accordance with the methods described herein. In a specific embodiment a heterologous nucleic acid encoding an oligosaccharyl transferase from C. jejuni is introduced into the host cell. In another specific embodiment, the oligosaccharyl transferase Pglb from C. jejuni is introduced into the host cells described herein.

In certain, more specific embodiments, a heterologous glycosylation operon is introduced into the host cells described herein. In certain embodiments the heterologous glycosylation operon possesses one or more mutations, i.e., one or more of the genes in the operon are mutated so as to inactive/delete the gene. In a specific embodiment a heterologous nucleic acid encoding the glycosylation operon from C. jejuni is introduced into the host cell.

### 5.2 Carrier Proteins

Any carrier protein suitable for use in the production of bioconjugates can be used herein. Exemplary carrier proteins include, without limitation, Exotoxin A of P. aeruginosa (EPA), CRM197, Diphtheria toxoid, tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit (CTB), cholera toxin, detoxified variants of cholera toxin, E. coli sat protein, the passenger domain of E. coli sat protein, C. jejuni AcrA, and C. jejuni natural glycoproteins.

In certain embodiments, the carrier proteins used in the generation of the bioconjugates described herein are modified, e.g., modified in such a way that the protein is less toxic and or more susceptible to glycosylation, etc. In a specific embodiment, the carrier proteins used in the generation of the bioconjugates described herein are modified such that the number of glycosylation sites in the carrier proteins is maximized in a manner that allows for lower concentrations of the protein to be administered, e.g., in an immunogenic composition, in its bioconjugate form. Accordingly in certain embodiments, the carrier proteins described herein are modified to include 1, 2, 3, 4, 5, 6, 7, 8, 9, 10, or more glycosylation sites than would normally be associated with the carrier protein (e.g., relative to the number of glycosylation sites associated with the carrier protein in its native/natural, e.g., “wild-type” state). In specific embodiments, introduction of glycosylation sites is accomplished by insertion of glycosylation consensus sequences anywhere in the primary structure of the protein. Introduction of such glycosylation sites can be accomplished by, e.g., adding new amino acids to the primary structure of the protein (i.e., the glycosylation sites are added, in full or in part), or by mutating existing amino acids in the protein in order to generate the glycosylation sites (i.e., amino acids are not added to the protein, but selected amino acids of the protein are mutated so as to form glycosylation sites). Those of skill in the art will recognize that the amino acid sequence of a protein can be readily modified using approaches known in the art, e.g., recombinant approaches, that include modification of the nucleic acid sequence encoding the protein. In specific embodiments, glycosylation consensus sequences are introduced into specific regions of the carrier protein, e.g., surface structures of the protein, at the N or C termini of the protein, and/or in loops that are stabilized by disulfide bridges at the base of the protein. In certain embodiments, the classical 5 amino acid glycosylation consensus sequence may be extended by lysine residues for more efficient glycosylation, and thus the inserted consensus sequence may encode 5, 6, or 7 amino acids that should be inserted or that replace acceptor protein amino acids.

In certain embodiments, the carrier proteins used in the generation of the bioconjugates described herein comprise a “tag,” i.e., a sequence of amino acids that allows for the isolation and/or identification of the carrier protein. For example, adding a tag to a carrier protein described herein can be useful in the purification of that protein and, hence, the purification of bioconjugates comprising the tagged carrier protein. Exemplary tags that can be used herein include, without limitation, histidine (HIS) tags (e.g., hexa histidine-tag, or 6XHis-Tag), FLAG-TAG, and HA tags. In certain embodiments, the tags used herein are removable, e.g., removal by chemical agents or by enzymatic means, once they are no longer needed, e.g., after the protein has been purified.

### 5.3 Modified E. coli O121 O-Antigens

The bioconjugates described herein comprise modified E. coli O121 O-antigens, wherein, as a result of modification of said antigens using the methods described herein (e.g., deletion of the wbqG gene), said modified E. coli O121 O-antigens contain structural similarities to the Salmonella enterica Vi polysaccharide, particularly the Salmonella enterica subspecies I serovar typhi (S. typhi) Vi polysaccharide. Without intending to be bound by theory, due to the similarity of the modified E. coli O121 O-antigens provided herein to the Salmonella enterica Vi polysaccharide, such modified E. coli O121 O-antigens are suitable for use in methods of treating and/or preventing infection of subjects (e.g., human subjects) by Salmonella enterica, particularly when said modified E. coli O121 O-antigens are administered as bioconjugates. Those of skill in the art will recognize, based on the discovery of the inventors, that any modified E. coli O121 O-antigens are suitable for use in accordance with the methods described herein, and can be used in the generation of the bioconjugates described herein, so long as said modified E. coli O121 O-antigen maintains similarity to the Salmonella enterica Vi polysaccharide, e.g., the Salmonella enterica subspecies I serovar typhi (S. typhi) Vi polysaccharide.

In a specific embodiment, provided herein is a modified E. coli O121 O-antigen, wherein said modified E. coli O121 O-antigen comprises the following structure:

→4)-α-D-GalNAcA-(1→4)-α-D-GalNAcA-(1→.

In another specific embodiment, provided herein is a modified E. coli O121 O-antigen, wherein said modified E. coli O121 O-antigen comprises the following structure:

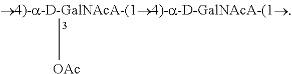

In another specific embodiment, provided herein is a modified E. coli O121 O-antigen, wherein said modified E. coli O121 O-antigen comprises the following structure:

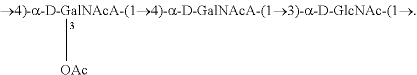

In another specific embodiment, provided herein is a modified E. coli O121 O-antigen, wherein said modified E. coli O121 O-antigen comprises the following structure:

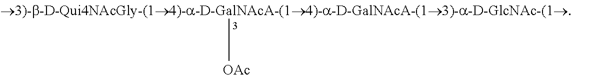

### 5.4 Bioconjugates

Provided herein are bioconjugates produced by the host cells described herein, wherein said bioconjugates comprise a carrier protein and a modified E. coli O121 O-antigen. As referred to herein, bioconjugates comprise a carrier protein and a modified E. coli O121 O-antigen, wherein said modified E. coli O121 O-antigen is covalently linked to an asparagine (ASN) residue of the carrier protein (e.g., linked at a glycosylation site of the carrier protein).

In a specific embodiment, provided herein is a bioconjugate comprising a carrier protein and a modified E. coli O121 O-antigen, wherein the modified E. coil O121 O-antigen comprises:

→4)-α-D-GalNAcA-(1→4)-α-D-GalNAcA-(1→.

In another specific embodiment, provided herein is a bioconjugate comprising a carrier protein and a modified E. coli O121 O-antigen, wherein the modified E. coli O121 O-antigen comprises:

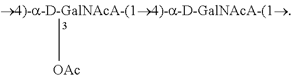

In another specific embodiment, provided herein is a bioconjugate comprising a carrier protein and a modified E. coli O121 O-antigen, wherein the modified E. coli O121 O-antigen comprises:

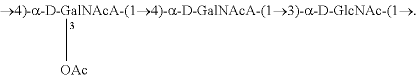

In another specific embodiment, provided herein is a bioconjugate comprising a carrier protein and a modified E. coli O121 O-antigen, wherein the modified E. coli O121 O-antigen comprises:

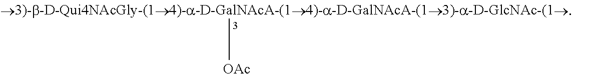

In certain embodiments, the bioconjugates provided herein are isolated, i.e., the bioconjugates are produced by a host cell described herein using methods of production of bioconjugates known in the art and/or described herein, and the produced bioconjugate is isolated and/or purified. In certain embodiments, the bioconjugates provided herein are at least 75%, 80%, 85%, 90%, 95%, 98%, or 99% pure, e.g., free from other contaminants, etc.

In certain embodiments, the bioconjugates provided herein are homogeneous with respect to the modified E. coli O121 O-antigen attached to the glycosylation sites of the carrier protein, e.g., the bioconjugates express the same modified E. coli O121 O-antigen at all glycosylation sites of the carrier protein.

In certain embodiments, the bioconjugates provided herein are not homogeneous with respect to the modified E. coli O121 O-antigens attached to the glycosylation sites of the carrier proteins, e.g., the bioconjugates express different modified E. coli O121 O-antigens, at the glycosylation sites of the carrier protein.

In certain embodiments, the bioconjugates provided herein possess greater than one glycosylation site, wherein each glycosylation site of the carrier protein is glycosylated (i.e., 100% of the glycosylation sites of the carrier protein are glycosylated), i.e., a modified E. coli O121 O-antigen is attached to each glycosylation site. In certain embodiments, the bioconjugates provided herein possess greater than one glycosylation site, wherein not all of the glycosylation sites of the carrier protein are glycosylated, e.g., about or at least 10%, 20%, 25%. 30%, 35%, 40%, 45%, 50%, 55%, 60%, 65%, 70%, 75%, 80%, 85%, 90%, or 95% of the glycosylation sites of the carrier protein are glycosylated, but not all of the glycosylation sites of the carrier protein are glycosylated (i.e., modified E. coli O121 O-antigens are not attached to each glycosylation site). In certain embodiments, all of the glycosylation sites of the carrier protein that are glycosylated comprise (i.e., are glycosylated with) the same modified E. coli O121 O-antigen.

In certain embodiments, provided herein are populations of bioconjugates. In one embodiment, provided herein is a population of bioconjugates, wherein at least 50%, 55%, 60%, 65%, 70%, 75%, 80%, 85%, 90%, or 95%, or wherein 100%, of a first glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the first glycosylation site of each bioconjugate is glycosylated with the same modified E. coli O121 O-antigen as the other bioconjugates in the population (i.e., all bioconjugates have the same modified E. coli O121 O-antigen at the first glycosylation site of the carrier protein). In certain embodiments, at least 50%, 55%, 60%, 65%, 70%, 75%, 80%, 85%, 90%, or 95%, or 100%, of a second glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the second glycosylation site of each bioconjugate is glycosylated with the same modified E. coli O121 O-antigen as the other bioconjugates in the population (i.e., all bioconjugates have the same modified E. coli O121 O-antigen at the second glycosylation site of the carrier protein). In certain embodiments, at least 50%, 55%, 60%, 65%, 70%, 75%, 80%, 85%, 90%, or 95%, or 100%, of a third glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the third glycosylation site of each bioconjugate is glycosylated with the same modified E. coli O121 O-antigen as the other bioconjugates in the population (i.e., all bioconjugates have the same modified E. coli O121 O-antigen at the third glycosylation site of the carrier protein). In certain embodiments, at least 50%, 55%, 60%, 65%, 70%, 75%, 80%, 85%, 90%, or 95%, or 100%, of a fourth glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the fourth glycosylation site of each bioconjugate is glycosylated with the same modified E. coli O121 O-antigen as the other bioconjugates in the population (i.e., all bioconjugates have the same modified E. coli O121 O-antigen at the fourth glycosylation site of the carrier protein). In certain embodiments, at least 50%, 55%, 60%, 65%, 70%, 75%, 80%, 85%, 90%, or 95%, or 100%, of a fifth glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the fifth glycosylation site of each bioconjugate is glycosylated with the same modified E. coli O121 O-antigen as the other bioconjugates in the population (i.e., all bioconjugates have the same modified E. coli O121 O-antigen at the fifth glycosylation site of the carrier protein).

### 5.5 Compositions

### 5.5.1 Compositions Comprising Host Cells

In one embodiment, provided herein are compositions comprising the host cells described herein. Such compositions can be used in methods for generating the bioconjugates described herein, e.g., the compositions can be cultured under conditions suitable for the production of proteins. Subsequently, the bioconjugates can be isolated from said compositions.

The compositions comprising the host cells provided herein can comprise additional components suitable for maintenance and survival of the host cells described herein, and can additionally comprise additional components required or beneficial to the production of proteins by the host cells, e.g., inducers for inducible promoters, such as arabinose, IPTG.

### 5.5.2 Compositions Comprising Bioconjugates

In another embodiment, provided herein are compositions comprising the bioconjugates described herein. Such compositions can be used in methods of treatment and prevention of disease. In a specific embodiment, the compositions described herein are used in the treatment of subjects (e.g., human subjects) infected with Salmonella enterica. In another specific embodiment, the immunogenic compositions described herein are used in the prevention treatment of subjects (e.g., human subjects) infected with Salmonella enterica subspecies I serovar typhi (S. typhi).

In a specific embodiment, provided herein are immunogenic compositions comprising one or more of the bioconjugates described herein. The immunogenic compositions provided herein can be used for eliciting an immune response in a host to whom the composition is administered. Thus, the immunogenic compositions described herein can be used as vaccines and can accordingly be formulated as pharmaceutical compositions. In a specific embodiment, the immunogenic compositions described herein are used in the prevention of infection of subjects (e.g., human subjects) by Salmonella enterica. In another specific embodiment, the immunogenic compositions described herein are used in the prevention of infection of subjects (e.g., human subjects) by Salmonella enterica subspecies I serovar typhi (S. typhi).

The compositions comprising the bioconjugates described herein may comprise any additional components suitable for use in pharmaceutical administration. In specific embodiments, the immunogenic compositions described herein are monovalent formulations. In other embodiments, the immunogenic compositions described herein are multivalent formulations. For example, a multivalent formulation comprises more than one bioconjugate described herein.

In certain embodiments, the compositions described herein additionally comprise a preservative, e.g., the mercury derivative thimerosal. In a specific embodiment, the pharmaceutical compositions described herein comprises 0.001% to 0.01% thimerosal. In other embodiments, the pharmaceutical compositions described herein do not comprise a preservative.

In certain embodiments, the compositions described herein (e.g., the immunogenic compositions) comprise, or are administered in combination with, an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a bioconjugate, but when the compound is administered alone does not generate an immune response to the bioconjugate. In some embodiments, the adjuvant generates an immune response to the poly bioconjugate peptide and does not produce an allergy or other adverse reaction. Adjuvants can enhance an immune response by several mechanisms including, e.g., lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998).

### 5.6 Uses

In one embodiment, provided herein are methods of treating an infection in a subject comprising administering to the subject a bioconjugate described herein or a composition thereof. In a specific embodiment, a method for treating an infection described herein comprises administering to a subject in need thereof an effective amount of a bioconjugate described herein or a composition thereof.

In another embodiment, provided herein are methods for inducing an immune response in a subject comprising administering to the subject a bioconjugate described herein or a composition thereof. In a specific embodiment, a method for inducing an immune response to a bioconjugate described herein comprises administering to a subject in need thereof an effective amount of a bioconjugate described herein or a composition thereof.

In a specific embodiment, the subjects to whom a bioconjugate or composition thereof is administered have, or are susceptible to, an infection, e.g., a bacterial infection. In another specific embodiment, the subjects to whom a bioconjugate or composition thereof is administered are infected with, or are susceptible to infection with Salmonella enterica. In another specific embodiment, the subjects to whom a bioconjugate or composition thereof is administered are infected with, or are susceptible to infection with Salmonella enterica subspecies I serovar typhi.

In another embodiment, the bioconjugates described herein can be used to generate antibodies for use in, e.g., diagnostic and research purposes, e.g., such antibodies are useful in determining whether administration of an immunogenic composition comprising a bioconjugate described herein, or any other composition used in the treatment of Salmonella enterica infection, results in a host immune response sufficient to kill or neutralize Salmonella enterica (e.g., such antibodies can be used in a serum bactericidal Assay).

### 5.7 Assays

### 5.7.1 Assay for Assessing Ability of Bioconjugates to Induce an Immune Response

The ability of the bioconjugates described herein to generate an immune response in a subject that is capable of cross-reacting with Vi polysaccharide of S. enterica can be assessed using any approach known to those of skill in the art or described herein. In some embodiments, the ability of a bioconjugate to generate an immune response in a subject that is capable of cross-reacting with Vi polysaccharide of S. enterica can be assessed by immunizing a subject (e.g., a mouse) or set of subjects with a bioconjugate described herein and immunizing an additional subject (e.g., a mouse) or set of subjects with a control (PBS). Such subjects can represent an animal model of disease, e.g., an animal model of typhoid fever (see, e.g., Libby et al., 2010, PNAS USA 107(35):15589-15594). The subjects or set of subjects can subsequently be challenged with a virulent S. enterica and the ability of the virulent S. enterica to cause disease (e.g., typhoid fever) in the subjects or set of subjects can be determined. Those skilled in the art will recognize that if the subject or set of subjects immunized with the control suffer from disease (e.g., typhoid fever) subsequent to challenge with the S. enterica but the subject or set of subjects immunized with bioconjugate described herein do not suffer from disease, then the bioconjugate is able to generate an immune response in a subject that is capable of cross-reacting with Vi polysaccharide of S. enterica. The ability of a bioconjugate described herein to induce antiserum that cross-reacts with Vi polysaccharide of S. enterica can be tested by, e.g., an immunoassay, such as an ELISA.

### 5.7.2 Serum Bactericidal Assay

The ability of the bioconjugates described herein to generate an immune response in a subject that is capable of cross-reacting with Vi polysaccharide of S. enterica can be assessed using a serum bactericidal assay (SBA). Such assays are well-known in the art and, briefly comprise the steps of generating and isolating antibodies against a target of interest (e.g., Vi polysaccharide of S. enterica) by administering to a subject (e.g., a mouse) a compound that elicits such antibodies. Subsequently, the bactericidal capacity of the antibodies can be assessed by, e.g., culturing the bacteria in question (e.g., S. enterica) in the presence of said antibodies and complement and assaying the ability of the antibodies to kill and/or neutralize the bacteria, e.g., using standard microbiological approaches.

### 6. EXAMPLES

This example demonstrates that modified E. coli O121 O-antigens can be successfully developed and that administration of bioconjugates comprising such antigens can elicit the production of antibodies in mice that are cross-reactive with the Vi polysaccharide of Salmonella enterica.

### 6.1 Materials and Methods

(a) Bacterial Strains, Plasmids, and Culture Conditions.

Bacterial strains and plasmids described in this example are listed in Table 1. Construction of the plasmids is described below. E. coli strains were grown in LB medium (10 g tryptone, 5 g yeast extract, and 5 g NaCl per liter) or LB agar (LB medium with the addition of 15 g agar per liter) at 37° C. S. Typhi BRD948 was grown in LB medium supplemented with 1% v/v Aro-mix (40 mg L-phenylalanine, 40 mg L-tryptophan, 10 mg 4-aminobenzoic acid, and 10 mg 2,3-dihydroxybenzoic acid in 10 ml of ddH₂O) and 1% v/v Tyr-mix (40 mg L-tyrosine disodium salt in 10 ml ddH₂O) at 37° C. If appropriate, the media contained tetracycline (20 μg ml⁻¹), spectinomycin (80 μg ml⁻¹), or ampicillin (100 μg ml⁻¹).

TABLE 1
Strains and plasmids used in this study.

| Strain | Genotype or relevant description | Reference |
|---|---|---|
| S. Typhi BRD948 | S. Typhi Ty2 ΔaroC aroD htrA | [Hone, et al., Vaccine |
| | | 9(11), 810-816 (1991)] |
| E. coli DH5a | K-12 φ80dlacZΔM15 endA1 recA1 hsdR17(rK-mK) supE44 thi-1 | Clonetech |
| | gyrA96 relA1 Δ(lacZYA-argF)U169 F- | |
| E. coli O121 | Escherichia coli serotype O121 | CCUG 11422* |
| SCM6 | SΦ874, Δwec waaL | C. Marolda and M. |
| | | Valvano, unpublished |
| W3110 | rph-1 IN(rrnD-rrnE)1 λ- | CGSC 4474** |
| Clm24 | W3110, ΔwaaL | [Feldman, et al., Proc |
| | | Natl Acad Sci USA |
| | | 102(8), 3016-3021 |
| | | (2005)] |
| Plasmid | Genotype or relevant description | |
| PLAFR1 | low copy-number broad host-range cosmid cloning vector; Tet^{r} | [Friedman, et al., Gene |
| | | 18(3), 289-296 (1982)] |
| PEXT21 | tac promoter expression vector; Sp^{r} | [Dykxhoorn, et al., |
| | | Gene 177(1-2), 133- |
| | | 136 (1996)] |
| Plasmid 1 | PLAFR1 derivative with multiple cloning site inserted in EcoRI site | This study |
| Plasmid 2 | Plasmid 1 derivative carrying O121 O antigen gene cluster of E. | This study |
| | coli CCUG11422 on an AscI/SpeI fragment; Tet^{r} | |
| Plasmid 3 | Plasmid 2 derivative containing inactivated wbqG | This study |
| Plasmid 4 | PEXT21 derivative carrying wecA, IPTG inducible, Sp^{r} | This study |
| PGVXN150 | Soluble periplasmic His₆-tagged toxoid variant (L552V, DE553) | [Ihssen, et al., Microb |
| | of P. aeruginosa exotoxin A (EPA) containing two engineered N- | Cell Fact 9, 61 (2010)] |
| | glycosylation sites cloned in pEC415, arabinose inducible, Amp^{R} | |
| PGVXN114 | HA-tagged pglB cloned in PEXT21, IPTG inducible, Sp^{r} | [Ihssen, et al., Microb |
| | | Cell Fact 9, 61 (2010)] |

*Culture Collection University of Göteborg, Curator: Prof. E. R. B. Moore, Göteborg, Sweden
**The Coli Genetic Stock Center, Yale University, New Haven, CT, USA

(b) DNA Manipulations

Plasmid DNA was isolated using the NucleoSpin Plasmid or NucleoBond Xtra Maxi Plus kit (Macherey-Nagel). Total chromosomal DNA was isolated using NucleoSpin Tissue kit (Macherey-Nagel). Restriction enzymes (Fermentas), shrimp alkaline phosphatase (Fermentas), T4 DNA ligase (Fermentas), and Phusion High-Fidelity DNA polymerase (Finnzyme) were used according to the manufacturer's instructions. PCR and restriction fragments were purified for cloning using the NucleoSpin Extract II kit (Macherey-Nagel). All DNA sequencing was completed by Synergene Biotech GmbH (Switzerland) and synthetic oligonucleotides were ordered at Microsynth AG (Switzerland).

(c) Plasmid Constructions

Plasmid 1 contains a synthetic oligonucleotide cassette formed from annealing of 5′-AATTGGCGCGCCCGGGACTAGTCTTGGG (SEQ ID NO.: 1) and 5′-AATTCCCAAGACTAGTCCCGGGCGCGCC (SEQ ID NO.: 2) ligated into the EcoRI-digested PLAFR1 [Friedman, A. M., Long, S. R., Brown, S. E., Buikema, W. J., Ausubel, F. M.: Construction of a broad host range cosmid cloning vector and its use in the genetic analysis of Rhizobium mutants. Gene 18(3), 289-296 (1982)], thereby introducing unique AscI and SpeI single restriction sites. The E. coli O121 O antigen cluster was amplified from genomic DNA prepared from E. coli O121 (CCUG 11422) using the primers 5′-AAAGGCGCGCCGCGAAGGTAAAGTCAGCCG (SEQ ID NO.: 3) and 5′-AAAACTAGTCAGGAGTGAATTAAGTCATTG (SEQ ID NO.: 4). The digested PCR fragment was ligated into the AscI/SpeI digested Plasmid 1 resulting in Plasmid 2. Plasmid 3 was constructed by inserting a synthetic oligonucleotide cassette formed from annealing of 5′-TGAATGAATGAACTAGTTCAATCACTCA (SEQ ID NO.: 5) and 5′-TGAGTGATTGAACTAGTTCATTCATTCA (SEQ ID NO.: 6) into the single restriction site PmlI, interrupting the open reading frame of wbqG.

(d) LPS Analysis

Cells of an overnight culture equivalent to an A₆₀₀ of 1 were collected, resuspended in 100 μl of 1× sample buffer according to Laemmli [Laemmli, Favre, M.: Maturation of the head of bacteriophage T4. I. DNA packaging events. J Mol Biol 80(4), 575-599 (1973)] and boiled at 95° C. for 10 min. Proteinase K (Fermentas) was added to a final concentration of 200 μg/ml and the sample was incubated at 60° C. for 1 h. The LPS molecular species from the proteinase K-digested whole cell lysates were separated by SDS-PAGE using a 12% BisTris NuPAGE gel from Invitrogen and MES running buffer according to manufacturer's instructions. LPS was visualized by staining with silver [Tsai, C. M., Frasch, C. E.: A sensitive silver stain for detecting lipopolysaccharides in polyacrylamide gels. Anal Biochem 119(1), 115-119 (1982)]. Immunological properties of O antigens were analyzed by Western blot using standard methods. The structure of the E. coil O121 O antigen is identical to the Shigella dysenteriae type 7 0 antigen therefore an anti- S. dysenteriae type 7 sera was purchased from Reagensia AB (Sweden) and used in a 1:100 dilution. Anti-Vi polyclonal antibody was purchased from Murex Biotech Ltd (England) and used in a 1:100 dilution.

(e) Analysis of Undecaprenyl Pyrophosphate (Und-PP)-linked O Antigen Glycans

The O antigen glycans were analyzed in E. coil strain SCM6, which contains chromosomal deletions in several polysaccharide gene clusters. The O polysaccharide was expressed by transforming SCM6 cells with a plasmid encoding the O antigen cluster and the wecA expression plasmid (Plasmid 4). SCM6 transformed with empty plasmids was used as a negative control to identify O antigen specific signals. The strains were grown overnight in a shake flask. Cells equivalent to an A₆₀₀ of 400 were harvested, washed once with 0.9% NaCl, and lyophilized. Lipids were extracted from the dried cells with 95% methanol (MeOH) by repeated rounds of vortexing and incubation on ice for 10 min. The suspension was converted into 85% MeOH by the addition of ddH₂O and further incubated for 10 min on ice while regularly vortexing. After centrifugation, the supernatant was collected and the extract was dried under N₂. The dried lipids were dissolved in 1:1 methanol/water (M/W) containing 10 mM tetrabutylammonium phosphate (TBAP) and subjected to a C₁₈ SepPak cartridge (Waters Corp., Milford, Mass.). The cartridge was conditioned with 10 ml MeOH, followed by equilibration with 10 ml 10 mM TBAP in 1:1 M/W. After loading of the sample, the cartridge was washed with 10 ml 10 mM TBAP in 1:1 M/W and eluted with 5 ml MeOH followed by 5 ml 10:10:3 chloroform/methanol/water (C/M/W). The combined elution fractions were dried under N₂.

The lipid samples were hydrolyzed according to Glover et al. [Glover, K. J., Weerapana, E., Imperiali, B.: In vitro assembly of the undecaprenylpyrophosphate-linked heptasaccharide for prokaryotic N-linked glycosylation. Proc Natl Acad Sci U S A 102(40), 14255-14259 (2005)] by dissolving the dried samples in 2 ml 1 M trifluoroacetic acid (TEA) in 50% n-propanol and heating to 50° C. for 15 min. The hydrolyzed samples were dried under N₂, dissolved in 4 ml 3:48:47 C/M/W and subjected to a C₁₈ SepPak cartridge (Waters Corp., Milford, Mass.) to separate the lipids from the hydrolyzed glycans. The cartridge was conditioned with 10 ml MeOH, followed by equilibration with 10 ml 3:48:47 C/M/W. The samples were applied to the cartridge and the flow-through was collected. The cartridge was washed with 4 ml 3:48:47 C/M/W and the combined flow-through fractions were dried using a SpeedVac.

The dried samples were labeled with 2-aminobenzamide (2AB) according to Bigge et al. [Bigge, J. C., Patel, T. P., Bruce, J. A., Goulding, P. N., Charles, S. M., Parekh, R. B.: Nonselective and efficient fluorescent labeling of glycans using 2-amino benzamide and anthranilic acid. Analytical biochemistry 230(2), 229-238 (1995)]. The glycan clean-up was performed using the paper disk method as described in Merry et al. [Merry, A. H., Neville, D. C., Royle, L., Matthews, B., Harvey, D. J., Dwek, R. A., Rudd, P. M.: Recovery of intact 2-aminobenzamide-labeled O-glycans released from glycoproteins by hydrazinolysis. Analytical biochemistry 304(1), 91-99 (2002)]. The separation of 2AB-labeled glycans was performed by HPLC using a GlycoSep N normal phase column according to Royle et al. [Royle, L., Mattu, T. S., Hart, E., Langridge, J. I., Merry, A. H., Murphy, N., Harvey, D. J., Dwek, R. A., Rudd, P. M.: An analytical and structural database provides a strategy for sequencing O-glycans from microgram quantities of glycoproteins. Analytical biochemistry 304(1), 70-90 (2002)], but modified to a three solvent system. Solvent A: 10 mM ammonium formate pH 4.4 in 80% acetonitrile. Solvent B: 30 mM ammonium formate pH 4.4 in 40% acetonitrile. Solvent C: 0.5% formic acid. The column temperature was 30° C. and 2AB-labeled glycans were detected by fluorescence (λex=330 nm, λem=420 nm). Gradient conditions: A linear gradient of 100% A to 100% B over 160 min at a flow rate of 0.4 ml min⁻¹, followed by 2 min 100% B to 100% C, returning to 100% A over 2 min and running for 15 min at 100% A at a flow rate of 1 ml min⁻¹, then returning the flow rate to 0.4 ml min⁻¹ for 5 min. samples were injected in ddH₂O.

To identify O antigen specific glycans, the 2AB glycan profile from cells carrying an empty plasmid control was subtracted from the trace obtained from cells harboring an O antigen cluster. The O antigen specific peaks were collected and 2AB glycans were analyzed on a MALDI SYNAPT HDMS Q-TOF system (Waters Corp., Milford, Mass.). Samples were dissolved in 5:95 acetonitrile/water and spotted 1:1 with 20 mg ml⁻¹ DHB in 80:20 methanol/water. Calibration was done with PEG (Ready mixed solution, Waters Corp., Milford, Mass.), spotted 1:3 with 5 mg ml⁻¹ α-cyano-4-hydroxycinnamic acid (CHCA, Sigma-Aldrich, Switzerland) in 60:40:0.1 acetonitrile/water/trifluoroacetic acid. The instrument was equipped with 200 Hz solid state UV laser. Mass spectra were recorded in positive ion mode. For MSMS: laser energy was fixed at 240 at a firing rate of 200 Hz, collision gas was argon. A collision energy profile was used to ramp collision energy depending on the m/z. Combined, background subtracted, and smoothened (Savitzsky Golay) spectra were centered using MassLynx v4.0 software (Waters Corp., Milford, Mass.).

(f) Production and Purification of Glycoconjugates

The production of glycoconjugates was achieved by expressing the oligosaccharyl transferase PglB, the engineered acceptor protein EPA (exotoxin A of Pseudomonas aeruginosa), and a gene cluster producing undecaprenyl-pyrophosphate (Und-PP)-linked glycans in E. coli. PGVXN114 (expressing PglB), PGVXN150 (expressing C-terminal His₆-tagged EPA) and Plasmid 2 (0121 antigen cluster) or Plasmid 3 (O121 wbqG mutant antigen) were co-transformed into E. coli strain Clm24 [Feldman, M. F., Wacker, M., Hernandez, M., Hitchen, P. G., Marolda, C. L., Kowarik, M., Morris, H. R., Dell, A., Valvano, M. A., Aebi, M.: Engineering N-linked protein glycosylation with diverse O antigen lipopolysaccharide structures in Escherichia coli. Proc Natl Acad Sci U S A 102(8), 3016-3021 (2005)]. Cells were cultured in LB medium supplemented with antibiotics at 37° C. in the shaker incubator (180 rpm). Shake flask expression cultures were inoculated from an uninduced overnight culture to an A₆₀₀ of 0.05. Expression of PglB and the carrier protein EPA was induced at an A₆₀₀ of 0.4-0.6 by IPTG (1 mM) and L-arabinose (0.02% w/v). Four hours after the first induction a second pulse of L-arabinose (0.02% w/v) was added. Cells were harvested after overnight incubation (total induction time of 19-22 h). Pellets were washed with 0.9% NaCl and suspended in resuspension buffer (25% sucrose, 10 mM EDTA, 200 mM Tris HCl pH 8.0) at a concentration equivalent to an A₆₀₀ of 50. The cell suspension was incubated on a shaker for 20 min at 4° C. After centrifugation the cell pellet was resuspended in the same volume of osmotic shock buffer (10 mM Tris HCl pH 8.0). The suspension was incubated on a shaker for 30 min at 4° C. and centrifuged at 10'000 g for 20 min to remove the spheroblasts. The supernatant containing periplasmic proteins was collected and the recombinant EPA containing a C-terminal hexahistidine tag was purified using a HisTrap crude FF 1 ml column (GE Healthcare, Switzerland). The extract was diluted with 5× HT binding buffer (2.5 M NaCl, 150 mM Tris HCl pH 8.0, 50 mM imidazole) to optimize the binding conditions and MgCl₂ was added to a final concentration of 50 mM. The extract was filtered and applied to the HisTrap crude FF column equilibrated with 1× HT binding buffer. After loading the column was washed with the same buffer containing 20 mM imidazole to remove unbound proteins. Proteins were eluted from the column with HT elution buffer (HT binding buffer containing 0.5 M imidazole).

Subsequently, the glycoprotein was separated from the unglycosylated EPA using a Resource Q 1 ml column (GE Healthcare, Switzerland). The HisTrap elution fractions containing EPA were pooled and diluted 10× with RQ binding buffer (20 mM L-histidine, pH 6.0). The diluted EPA sample was applied to the anion exchange column equilibrated with RQ binding buffer. The column was eluted with a linear gradient from 0% to 32.5% of RQ elution buffer (RQ binding buffer containing 1 M NaCl) in 25 column volumes and 0.5 ml fractions were collected using an Äkta FPLC (Amersham Biosciences). The fractions were analyzed by SDS-PAGE and proteins were stained with Coomassie blue. Fractions containing glycoprotein were pooled and buffer was exchanged to PBS using an Amicon Ultra-4 centrifugal filter unit with a 30 kDa membrane (Millipore) by performing several concentration and dilution steps according to manufacturer's instructions. The concentration of the final purified protein sample was adjusted to 1 mg ml⁻¹.

(g) Purification of E. coli O121 LPS

LPS of an E. coli O121 (CCGU 11422) culture was purified by phenol extraction as described elsewhere [Apicella, M. A.: Isolation and characterization of lipopolysaccharides. Methods Mol Biol 431, 3-13 (2008)].

(h) Purification of Vi Polysaccharide and Modification with Tyramine

Vi polysaccharide was purified from S. Typhi BRD948 by a modified procedure as previously described [Demil, P., D'Hondt, E., Hoecke, C. V.: Salmonella Typhi vaccine compositions. European Patent EP1107787 (2003)]. Briefly, S. Typhi BRD948 was grown in LB medium supplemented with Aro- and Tyr-mix. After overnight incubation at 37° C. in the shaker incubator (180 rpm) the culture was heated to 60° C. for 1 h and centrifuged. Vi was precipitated from the supernatant with 0.1% hexadecyltrimethylammonium bromide (CTAB, Sigma, H6269). 20 g 1⁻¹ celite 545 (Sigma, 20199-U) was added and the mixture was stirred for 1 h at room temperature (RT) in order to allow the formation of a polysaccharide-CTAB complex, which adsorbs onto the celite. The celite was poured into a reservoir of appropriate size (Extract-clean EV SPE Reservoir, Socochim S. A.) equipped with a frit (Socochim S. A.). The column was washed successively by gravity flow with 10 column volumes (CV) of 0.05% CTAB, 10 CV of 20% ethanol, 50 mM sodium phosphate buffer pH 6.0, and 14 CV of 45% ethanol to eliminate adsorbed impurities. The Vi polysaccharide was finally eluted with 1.5 CV of 50% ethanol, 0.4 M NaCl. Following elution, the polysaccharide was precipitated by the addition of ethanol to a final concentration of 80% and incubation for 20 min at RT. Finally, the precipitated polysaccharide was collected by centrifugation for 20 min at 15000 g, washed twice with 80% ethanol, and lyophilized.

The protein and nucleic acid content of the purified Vi polysaccharide was determined by the bicinchoninic acid assay (BCA) and UV spectroscopy respectively. O-acetyl content was measured with acetylcholine as standard [Hestrin, S.: The reaction of acetylcholine and other carboxylic acid derivatives with hydroxylamine, and its analytical application. J Biol Chem 180(1), 249-261 (1949)].

To increase the binding efficiency of the Vi to microtiter plates, the polysaccharide was tyraminated (Vi-Tyr). Tyramine hydrochloride (30 mg ml⁻¹, Sigma) was added to 10 mg of purified Vi. 100 μl of 0.5 M N-(3-dimethylaminopropyl)-N′-ethylcarbodiimide HCl (Sigma) was added and the mixture was incubated at pH 4.9-5.1 for 3 h. The reaction mixture was dialyzed against ddH₂O.

(i) Immunization Studies

Groups of 7 CB6F1 female mice, 6-8 weeks old, were used in immunization experiments. Mice were immunized, subcutaneously, with 20 μg of glycoconjugate with Alum (Rehydragel LV—Aluminium Hydroxide, General Chemical) as adjuvant or 5 μg of Vi polysaccharide (Typhim Vi, Sanofi Pasteur MSD). Adjuvantation of the glycoconjugate was done just before immunization. Briefly, the purified glycoconjugates were diluted with PBS to a final concentration of 200 μg ml⁻¹, Alum (final amount of Al³⁺ corresponded to 0.6 mg ml⁻¹) was added, and the solution was gently mixed for 1 h at room temperature. Immunizations were performed on days 1, 22 and 57. Groups of mice normally received 100 μl doses of vaccines, corresponding to 20 μg of conjugate (protein). Blood samples were collected 10 days after the second and 10 days after the last immunization.

(j) Enzyme-linked Immunosorbent Assay (ELISA)

Flat bottom 96 well micro-titer plates (Nunc immuno PolySorb) were coated with 50 μl of 5 μg ml⁻¹ E. coli O121 LPS or 5 μg ml⁻¹ of tyraminated Vi (Vi-Tyr), diluted in PBS, at 4° C. overnight. The coating solution was poured away and the plate was submerged and vigorously agitated in 4000 ml of wash buffer (1× PBS with 0.05% Triton×100). This washing step was performed at least 4 times. Subsequently, the plate was dried by placing and spinning upside down in a micro plate rotor. This washing procedure was always applied in further washing steps. Each well was completely filled with 300 μl of blocking buffer (1× PBS with 2.5% BSA (globulin free BSA, Sigma, A7030)) and incubated 2 h at room temperature (RT) on a plate shaker. After washing and drying the plate, dilutions of mouse serum in dilution buffer (1× PBS with 0.5% BSA) were added to the plate (100 μl) and incubated 1 h at RT on a plate shaker. To detect total immunoglobulin (Ig), 100 μl of horseradish peroxidase (HRP) labeled goat anti-mouse Ig (Sigma) diluted 1:2000 in dilution buffer was added to each well and the plate was incubated for 1 h at RT on a plate shaker. Following washing and drying the plate, the reaction was developed with 100 μl of Ultra TMB substrate (3,3′,5,5′-tetramethybenzidine liquid substrate, Pierce) for 15 min and stopped with the addition of 100 μl of 2 M sulphuric acid. Optical density (OD) was measured at 450 nm.

To determine the endpoint titer a 95% confidence level was defined according to [Frey, A., Di Canzio, J., Zurakowski, D.: A statistically defined endpoint titer determination method for immunoassays. J Immunol Methods 221(1-2), 35-41 (1998]. As negative sample a pool of preimmune sera was used.

### 6.2 RESULTS

(a) Analysis of the E. coli O121 wbqG Mutant O Polysaccharide

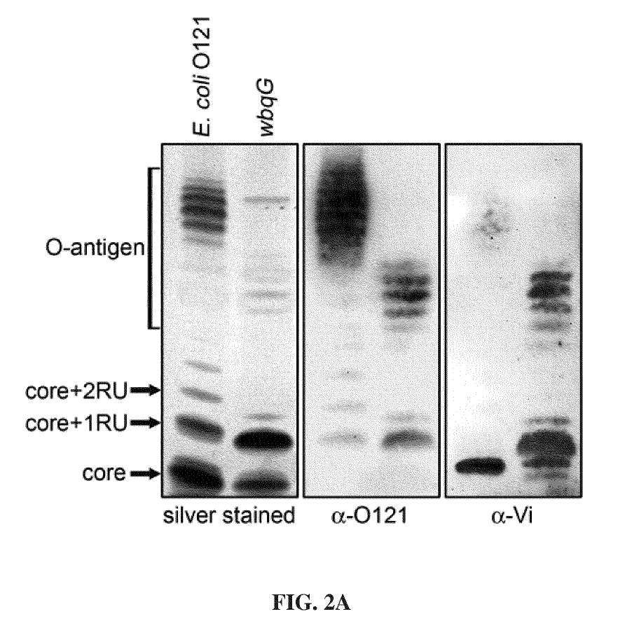

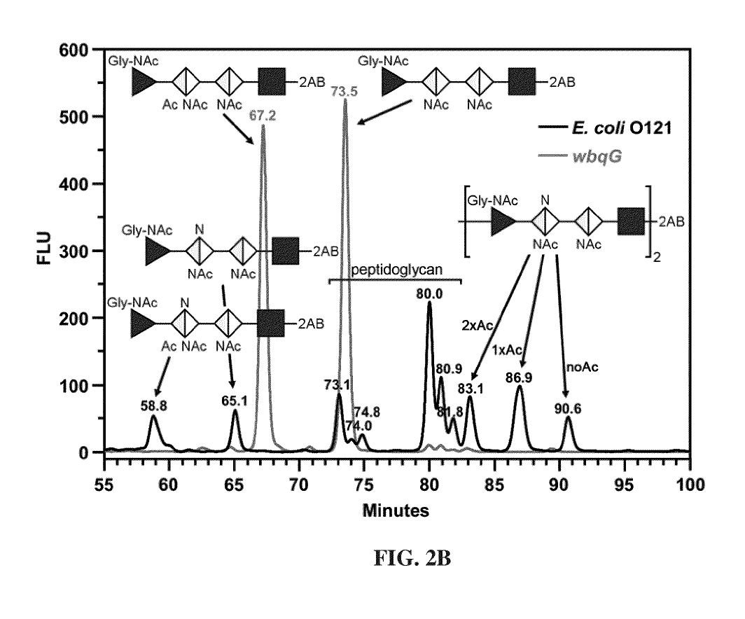

Whether the O polysaccharide produced by an E. coli O021 wbqG mutant would be recognized by antibodies specific for the Salmonella Typhi Vi capsular polysaccharide was first examined. The E. coli O121 O antigen gene cluster was cloned, and the open reading frame of wbqG was interrupted by insertion of a STOP codon containing oligocassette. The cloned plasmids were transformed into the E. coli K-12 strain W3110 and the lipopolysaccharide (LPS) was analyzed by SDS-PAGE and staining with silver, or after transferring to a nitrocellulose membrane by Western blot (FIG. 2A). As previously reported, mutation of the wbqG gene did not abolish O antigen expression [King, J. D., Vinogradov, E., Tran, V., Lam, J. S.: Biosynthesis of uronamide sugars in Pseudomonas aeruginosa O6 and Escherichia coli O121 O antigens. Environ Microbiol 12(6), 1531-1544 (2010)]. However, the LPS profile of the wbqG mutant visualized in the silver-stained polyacrylamide gel differed from the wild type in several points: (i) the staining of polymerized O antigen containing bands is fainter relative to wild type LPS, (ii) the band consisting of one O antigen repeating unit attached to the lipid A-core (core+1 RU) stained more intensely and (iii) the O antigen containing bands migrated faster than the equivalent bands of the wild type LPS. It was estimated that both LPS profiles contained an average of 12 O antigen repeat units attached to the lipid A-core by analyzing an overexposed silver-stained SDS-PAGE gel. Western blot analysis of the LPS revealed that anti-O121 sera reacted with the wbqG mutant O antigen. The wbqG mutant O polysaccharide was recognized by anti-Vi serum.

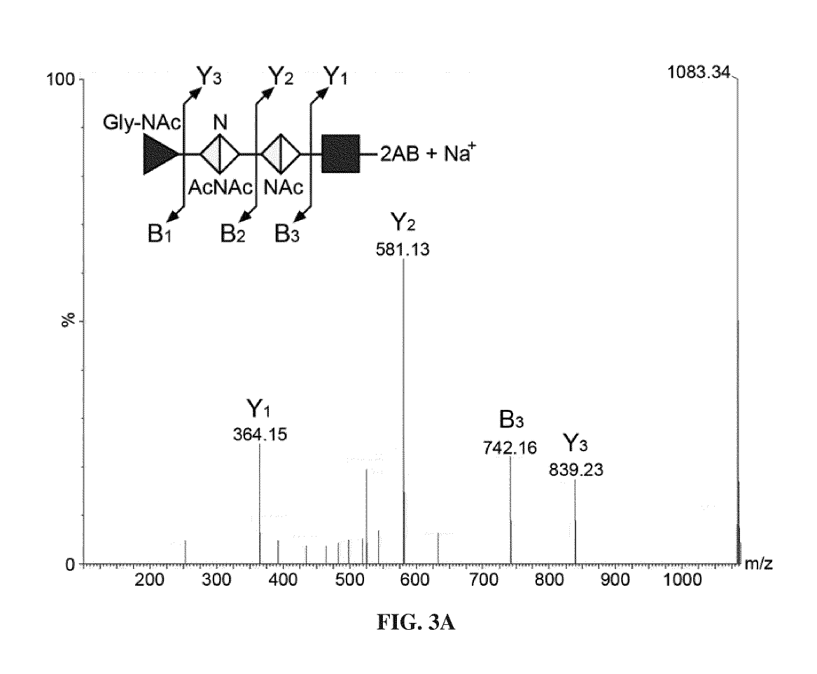

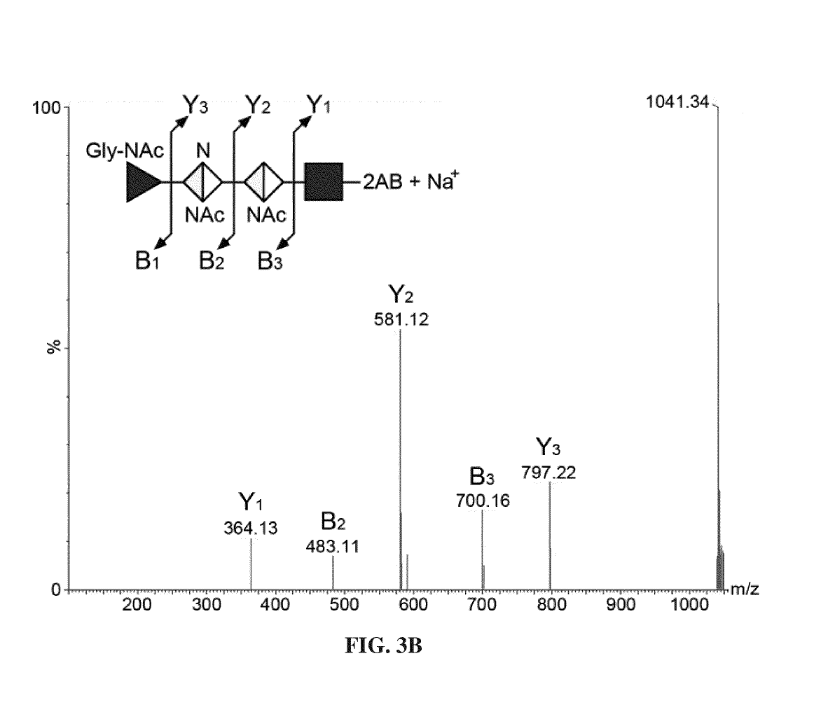

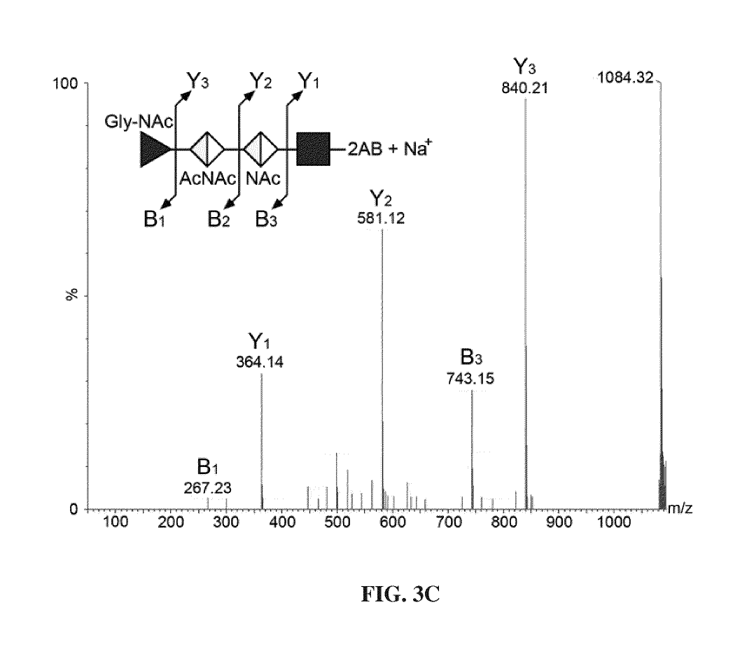

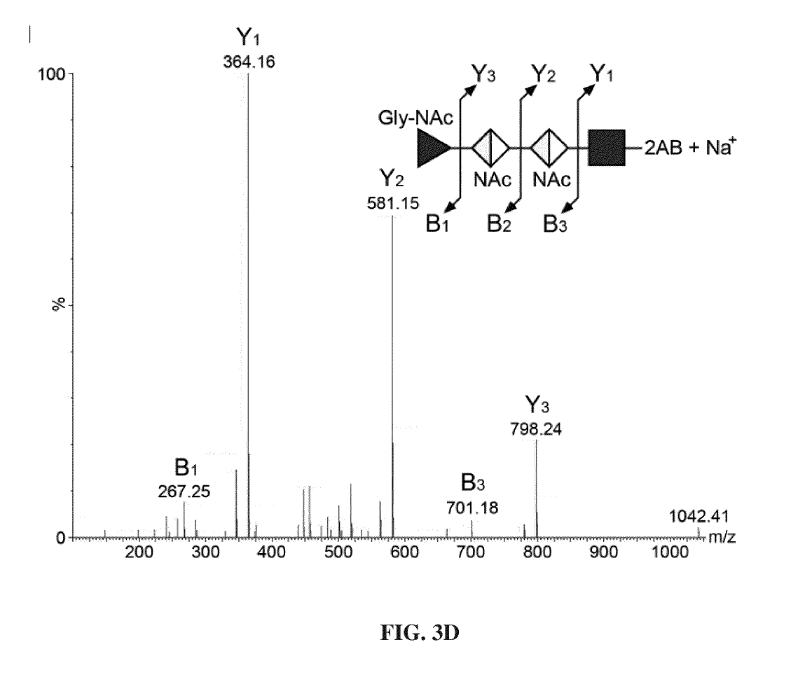

In order to confirm the structure of the expressed O antigen repeat unit and to determine the degree of O-acetylation, glycolipids were extracted from E. coli SCM6 strains expressing either the O121 wild type or the wbqG mutant O antigen. The lipid-linked oligosaccharides were purified using a C₁₈ SepPak column and treatment with mild acid specifically released Und-PP-linked glycans. After an additional purification step using again a C₁₈ SepPak column, the glycans were labeled with 2-aminobenzamide (2AB) and subsequently resolved by normal phase HPLC using a GlycoSep N column. FIG. 2B shows a section of the chromatogram where single repeat units and short polymerized O antigens are expected to elute. Fractions containing putative 2AB-labeled glycan species were analyzed by mass spectrometry (MS) (FIG. 3), and the glycan structures identified by MS are illustrated in FIG. 2B.

The chromatogram of the 2AB-labeled glycans prepared from SCM6 cells expressing the O121 wild type O antigen, featured a peak eluting at 58.8 min. In this peak fraction a molecule with a mass-to-charge ratio (m/z) of 1083 was identified. The peak fraction with the retention time of 65.1 min contained mainly a species with m/z of 1041. This detected m/z corresponded to the single-charged sodium adduct of a 2AB-labeled, non-acetylated O121 wild type subunit. The difference between the two detected masses corresponded to 42 Da, which is the mass difference between an O-acetyl and a hydroxyl group. These two species were subjected to collisionally induced dissociation (CID) MS-MS. The series of single-charged fragment ions obtained from the precursor with m/z of 1083 (FIG. 3A) was consistent with glycosidic cleavage products from the 2AB-labeled O121 wild type O antigen repeat unit, containing an O-acetyl group at residue c. Whereas the CID MS-MS spectra of the molecular species with m/z of 1041 corresponded to the non-acetylated 2AB-labeled O121 subunit (FIG. 3B).

The chromatogram of the 2AB-labeled glycans prepared from SCM6 cells expressing the wbqG mutant polysaccharide revealed two prominent peaks. In the peak fraction with the retention time of 67.2 min a molecule with m/z of 1084 was detected. This measured mass differed by 1 Da from the mass measured in the corresponding peak of the O121 wild type trace eluting at 58.8 min. Likewise an m/z of 1042 was measured for the 2AB-labeled molecule with a retention time of 73.5 min. CID MS-MS of these precursor ions (FIG. 3C and 3D) resulted in a fragmentation pattern that resembled the spectra obtained from the O121 wild type 2AB-labeled glycans. The measured mass difference of 1 Da was assigned to residue c of the glycan structure. The mass difference of 1 Da corresponded to the calculated mass difference between an acid and an amide group, in agreement with the published structure of the wbqG mutant O antigen [King, J. D., Vinogradov, E., Tran, V., Lam, J. S.: Biosynthesis of uronamide sugars in Pseudomonas aeruginosa O6 and Escherichia coli O121 O antigens. Environ Microbiol 12(6), 1531-1544 (2010)].

Polymerized 2AB-labeled O antigen subunits were identified in the O121 wild type trace. Two subunits variably O-acetylated were identified in the peak fractions with the retention times 83.1 min, 86.9 min and 90.6 min respectively. The double acetylated species eluted first followed by the single acetylated and non-acetylated form. Due to the separation of the acetylated and non-acetylated forms, the degree of O-acetylation could be determined. In both strains approximately 50% of the single repeating units were O-acetylated.

Precursors of the peptidoglycan monomer were also identified in some peaks of the O121 wild type trace (FIG. 2B). Peptidoglycan precursors are also assembled on undecaprenyl pyrophosphate and are expected to be purified and labeled with the method used for O antigen subunits.

(b) Production of Glycoconjugates

The structure of the O121 wbqG mutant was confirmed, and it was shown to be cross-reactive with antibodies raised against the Vi antigen. Next, whether the wbqG mutant O polysaccharide could elicit antibodies that bind to the Vi was determined. Glycoconjugates were prepared for immunization studies. Glycoproteins were produced by expressing the bacterial oligosaccharyl transferase PglB, the engineered periplasmic carrier protein EPA (toxoid recombinant Pseudomonas aeruginosa exotoxin A), and either the E. coli O121 wild type or the wbqG mutant antigen in the E. coli K12 derivative CLM24 [Feldman, M. F., Wacker, M., Hernandez, M., Hitchen, P. G., Marolda, C. L., Kowarik, M., Morris, H. R., Dell, A., Valvano, M. A., Aebi, M.: Engineering N-linked protein glycosylation with diverse O antigen lipopolysaccharide structures in Escherichia coli. Proc Natl Acad Sci U S A 102(8), 3016-3021 (2005)]. Strain CLM24 lacks the O antigen ligase (WaaL). Therefore, the transfer of O antigen to lipid A-core is blocked and the Und-PP-linked O antigen substrate accumulates at the periplasmic face of the inner membrane providing the O antigen donor for the PglB-catalyzed transfer to specific asparagine residues within the protein acceptor. Additionally, E. coli K12 derivatives lack a functional endogenous O antigen gene cluster [Liu, D., Reeves, P. R.: Escherichia coli K12 regains its O antigen. Microbiology 140 (Pt 1), 49-57 (1994); Feldman, M. F., Marolda, C. L., Monteiro, M. A., Perry, M. B., Parodi, A. J., Valvano, M. A.: The activity of a putative polyisoprenol-linked sugar translocase (Wzx) involved in Escherichia coli O antigen assembly is independent of the chemical structure of the O repeat. J Biol Chem 274(49), 35129-35138 (1999)]. A plasmid encoded O antigen gene cluster can therefore be expressed without producing mixed O antigen populations. As described elsewhere, EPA was used as protein acceptor with a N-terminal signal sequence for Sec-dependent secretion to the periplasm, and a C-terminal hexahistidine tag for purification by affinity chromatography [Ihssen, J., Kowarik, M., Dilettoso, S., Tanner, C., Wacker, M., Thony-Meyer, L.: Production of glycoprotein vaccines in Escherichia coli. Microb Cell Fact 9, 61 (2010)]. EPA contained two engineered N-glycosylation sites. The low copy plasmid PGVXN114 was used for the expression of PglB under the control of the IPTG inducible tac promoter.

After induction of PglB and EPA, the newly synthesized glycoprotein was purified from periplasmic extracts by nickel affinity chromatography. Due to the presence of negatively charged polysaccharides in the glycoconjugate, anion exchange chromatography was used to separate the glycosylated from the non-glycosylated forms. Based on the separation of the two species it was found that in cultures expressing the O121 wild type O polysaccharide gene cluster approximately 70% of the total EPA was glycosylated. The glycosylation efficiency was lower in cultures expressing the wbqG mutant O antigen whereas 35% of the total carrier protein contained the glycan modification.

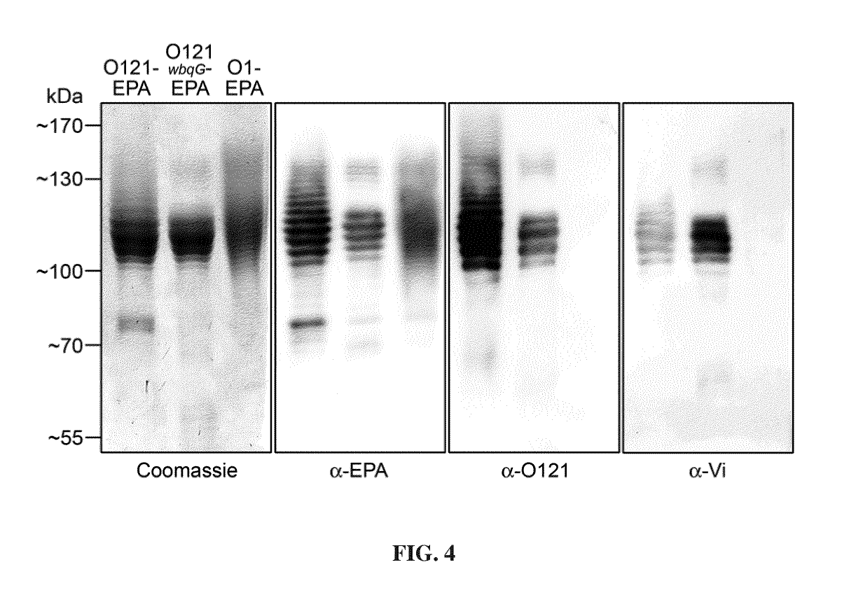

The purified glycoconjugates were separated by SDS-PAGE and visualized by Coomassie blue staining or by Western blot after transfer to a nitrocellulose membrane using anti-EPA, anti-O121, and anti-Vi antibodies (FIG. 4). By Coomassie blue staining a hand of the same mass as that of unglycosylated EPA (70 kDa) could be detected in the purified O121 polysaccharide-EPA conjugate (O121-EPA), that is also recognized by the anti-EPA but not the anti-O121 sera. Therefore, unglycosylated EPA was largely removed in the glycoconjugate preparations. Mainly, a ladder of bands clustered between 100 and 130 kDa was detected by Coomassie blue staining. These bands reacted with anti-EPA serum, indicating modified forms of EPA. These larger polypeptides, but not EPA modified with the Shigella dysenteriae O1 antigen (O1-EPA) (described in [Ihssen, J., Kowarik, M., Dilettoso, S., Tanner, C., Wacker, M., Thony-Meyer, L.: Production of glycoprotein vaccines in Escherichia coli. Microb Cell Fact 9, 61 (2010)]), were also detected with anti-O121 specific antibodies indicating the modification of the carrier with the co-expressed polysaccharide. EPA glycosylated with the wbqG mutant O polysaccharide (O121_{wbqG}-EPA) was additionally stained with anti-Vi antibodies.

As determined by SDS-PAGE analysis, mainly mono-glycosylated EPA was purified, i.e. EPA modified on one of the two engineered glycosylation sites with the corresponding O-polysaccharide. Traces of di-glycosylated EPA could be detected by western blot in the purified O121_{wbqG}-EPA sample (FIG. 4). The di-glycosylated form of EPA runs as a second fainter ladder of bands slightly bigger than 130 kDa. As seen in FIG. 2A, the expressed O-antigens display a modal chain length distribution with an average of 12 repeating units. Assuming the purified glycoconjugates consisted of mono-glycosylated EPA, containing a single polysaccharide chain of an average length of 12 repeating units, the sugar-to-protein weight ratio was estimated to be 0.15:1.

(c) Immunogenicity of the Glycoconjugates in Mice and Evaluation of the Polysaccharide Specific Antibody Response

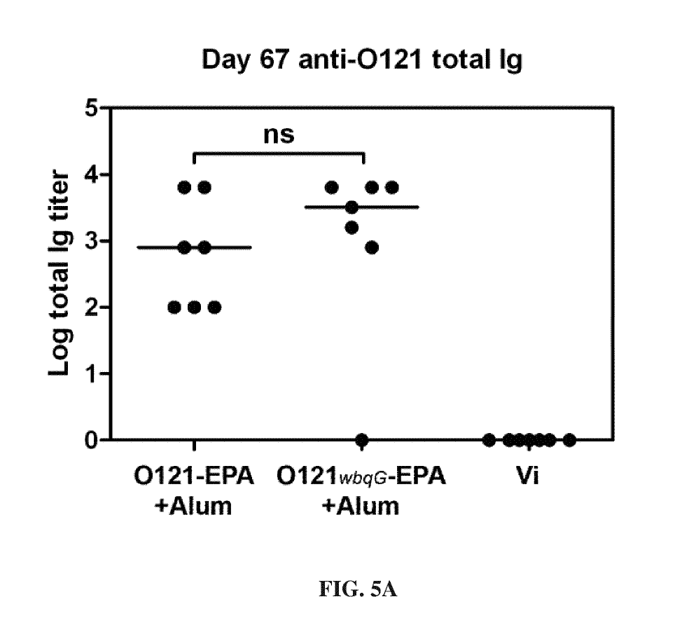

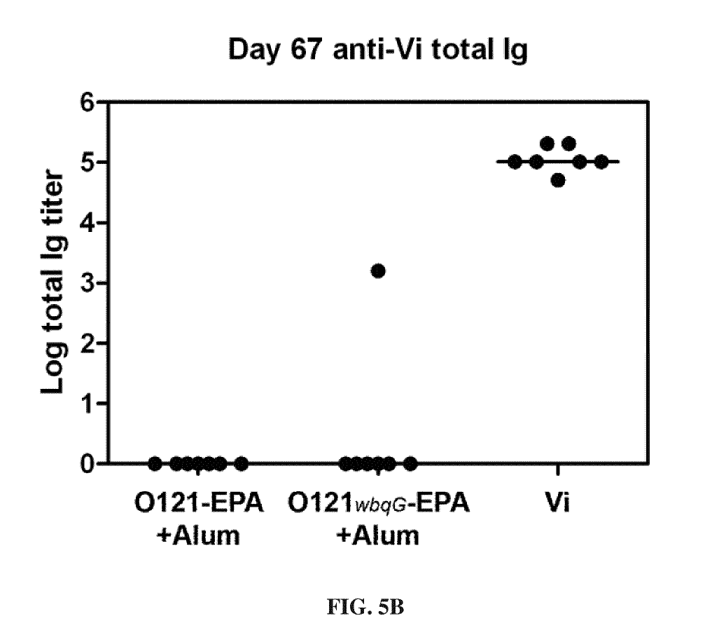

Next, the immune response elicited in mice upon immunization with the conjugate vaccines was assessed. Pilot experiments were conducted in small groups of CB6F1 mice to determine the dose range and adjuvantation of the purified glycoconjugates. These established that 20 μg of protein (approximately 3 μg of polysaccharide), in combination with Alum, were reproducibly immunogenic. Subsequently, groups of CB6F1 mice (7 per group) were immunized subcutaneously on days 1, 22 and 57 with O121-EPA, O121_{wbqG}-EPA, or with 5 μg of purified Vi polysaccharide (Typhim Vi, Sanofi Pasteur MSD). Mice were sample bled on days 32 and 67 and the sera were tested for the presence of anti-O121 LPS and anti-Vi total immunoglobulin (Ig). By day 67, a significant rise in serum Ig anti-O121 LPS titer was observed in 13 of 14 animals immunized with either conjugate (FIG. 5A). One animal in the group of mice that were immunized with O121_{wbqG}-EPA did not show seroconvertion. Interestingly, the same animal developed a significant rise in serum Ig anti-Vi titer (FIG. 5B). As expected, the control group that was immunized with purified Vi polysaccharide did not show a detectable anti-O121 LPS response but a significant rise in serum Ig anti-Vi titer.

### 6.3 Discussion

This example describes a novel method for the analysis of undecaprenyl pyrophosphate (Und-PP)-linked glycans. The procedure described is based on the method used to analyze dolichyl pyrophosphate (Dol-PP)-linked oligosaccharides of eukaryotic cells. Main modifications include an optimized extraction procedure for bacterial glycolipids and a purification step prior to glycan release by mild acid hydrolysis. The purification strategy of bacterial Und-PP-linked glycans is further complicated by the vast variety of different sugar structures assembled on this lipid carrier. The choice of an appropriate expression strain used to analyze a specific subclass of Und-PP-linked glycans is crucial. In this example, Und-PP-linked O polysaccharides were analyzed. Since Und-PP-linked O antigens represent an intermediate species of LPS biosynthesis, an E. coli strain was used lacking the O antigen ligase(ΔwaaL). Therefore, Und-PP-linked O polysaccharides are not transferred to lipid A-core, resulting in accumulations of this lipid intermediate. If O antigens were expressed in a waaL positive strain no 2AB-labeled O glycans could be identified, most likely due to the rapid turnover of this glycolipid species. Furthermore, O antigens are polymerized structures with high molecular weights, making it increasingly difficult for analysis by mass spectrometry. A strain background containing a mutation in the O antigen chain length regulator (wzz) gene involved in efficient polymerization of O antigen subunits was therefore chosen. This resulted in the production of mainly single repeat units and short polymerized O antigens, hence simplifying MS analysis. Several other polysaccharide structures are also assembled on Und-PP, like peptidoglycan precursors, capsular polysaccharides and the enterobacterial common antigen (ECA), which might complicate the identification and characterization of O glycan species. An E. coli strain, SCM6, which contains deletions in all major polysaccharide gene clusters was this used for O antigen expression.

With this modified method the O121 wbqG mutant O polysaccharides was analyzed. This example confirms the published structure by King et al. [King, J. D., Vinogradov, E., Tran, V., Lam, J. S.: Biosynthesis of uronamide sugars in Pseudomonas aeruginosa O6 and Escherichia coli O121 O antigens. Environ Microbiol 12(6), 1531-1544 (2010)]. Furthermore, it was determined that the recombinant expressed wbqG mutant O antigen structure contained O-acetylated N-acetylgalactosaminuronic acid, most likely modified at C-3. Therefore this mutant O polysaccharide contains structural motifs also present in the Vi polysaccharide. O-acetyl groups of the Vi polysaccharide form an immunodominant epitope and immunogenicity of Vi is closely related to the degree of O-acetylation [Szu, S. C., Bystricky, S.: Physical, chemical, antigenic, and immunologic characterization of polygalacturonan, its derivatives, and Vi antigen from Salmonella typhi. Methods Enzymol 363, 552-567 (2003); Szu, S. C., Li, X. R., Stone, A. L., Robbins, J. B.: Relation between structure and immunologic properties of the Vi capsular polysaccharide. Infect Immun 59(12), 4555-4561 (1991)].

This example shows for the first time that the wbqG mutant O polysaccharide is cross-reactive with antibodies raised against the Vi antigen.

Similarly, glycoconjugates composed of the E. coli O121 wild type or the wbqG mutant O polysaccharide and the P. aeruginosa exotoxin A (O121-EPA/O121_{wbqG}-EPA) were prepared in this example. EPA has already been successfully used as immunogenic carrier in a typhoid conjugate vaccine [Szu, S. C., Taylor, D. N., Trofa, A. C., Clements, J. D., Shiloach, J., Sadoff, J. C., Bryla, D. A., Robbins, J. B.: Laboratory and preliminary clinical characterization of Vi capsular polysaccharide-protein conjugate vaccines. Infect Immun 62(10), 4440-4444 (1994)]. Both groups of mice immunized with glycoconjugates developed glycan specific antibody responses. 6 of 7 mice immunized with the O121_{wbqG}-EPA conjugate showed a significant rise in serum immunoglobulin (Ig) anti-O121 LPS titer, indicating that other antigenic determinants than the uronamide groups are important for inducing an anti-O121 LPS specific immune response. Antibodies of one animal immunized with the O121_{wbqG}-EPA conjugate were not reactive with the E. coli O121 LPS but rather with the Vi polysaccharide. This indicates that this animal developed an antibody response against the epitope constituted by residues b and c′ (FIG. 1), which resembles the Vi structure. However, the other animals of this group raised antibodies against an O121-LPS specific epitope, most likely residue d, containing a prominent surface exposed side group. Further optimizations of the O121 glycan structure will improve the Vi specific immune response upon immunization.

TABLE 2
SEQUENCE LISTING

| SEQ ID NO. | Description | Sequence |
|---|---|---|
| SEQ ID NO: 1 | synthetic | AATTGGCGCGCCCGGGACTAG |
| | oligonucleotide | TCTTGGG |
| SEQ ID NO: 2 | synthetic | AATTCCCAAGACTAGTCCCGG |
| | oligonucleotide | GCGCGCC |
| SEQ ID NO: 3 | synthetic | AAAGGCGCGCCGCGAAGGTAA |
| | oligonucleotide | AGTCAGCCG |
| SEQ ID NO: 4 | synthetic | AAAACTAGTCAGGAGTGAATT |
| | oligonucleotide | AAGTCATTG |
| SEQ ID NO: 5 | synthetic | TGAATGAATGAACTAGTTCAA |
| | oligonucleotide | TCACTCA |
| SEQ ID NO: 6 | synthetic | TGAGTGATTGAACTAGTTCAT |
| | oligonucleotide | TCATTCA |

The embodiments described herein are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims.

All references (including patent applications, patents, and publications) cited herein are incorporated herein by reference in their entirety and for all purposes to the same extent as if each individual publication or patent or patent application was specifically and individually indicated to be incorporated by reference in its entirety for all purposes.

What is claimed is:

1. A bioconjugate comprising a carrier protein and a modified E. coli O121 O-antigen.

2. The bioconjugate of claim 1, wherein the modified E. coli O121 O-antigen is covalently hound to the Asn within a glycosylation site of the carrier protein wherein the glycosylation site comprises the amino acid sequence Asp/Glu-X-Asn-Z-Ser/Thr wherein X and Z may be any amino acid except Pro.

3. The bioconjugate of claim 2, wherein the glycosylation site has been recombinantly engineered and does not exist in the native carrier protein.

4. The bioconjugate of claim 2, wherein the carrier protein comprises 2, 3, 4, 5, 6, 7, 8, 9, or 10 glycosylation sites each having the amino acid sequence Asp/Glu-X-Asn-Z-Ser/Thr wherein X and Z may be any amino acid except Pro.

5. The bioconjugate of claim 1, wherein the carrier protein is selected from the group consisting of Exotoxin A of P. aeruginosa, CRM197, Diphteria toxoid, tetanus toxoid, detoxified hemolysin A of S. aureus, clumping factor A, clumping factor B, E. coli Firra, E. coli FimHC, E. coli heat labile enterotoxin, detoxified variants of E. coli heat labile enterotoxin, Cholera toxin B subunit, cholera toxin, detoxified variants of cholera toxin, E. coli sat protein, the passenger domain of E. coli sat protein, C. jejuni AcrA, and a C. jejuni natural glycoprotein, Neisseria meningitidis pilin, NMB0088, nitrite reductase (AniA), heparin-binding antigen (NHBA), factor H binding protein (fHBP), adhesin NadA, Ag473, surface protein A (NapA), an antigen of Salmonella enterica.

6. The bioconjugate of claim 1, wherein the modified E. coli O121 O-antigen comprises:
→4)-α-D-GalNAcA-(1→4)-α-D-GalNAcA-(1→.

7. The bioconjugate of claim 1, wherein the modified E. coli O121 O-antigen comprises:

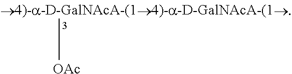

8. The bioconjugate of claim 1, wherein the modified E. coli O121 O-antigen comprises:

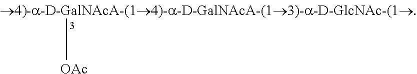

9. The bioconjugate of claim 1, wherein the modified E. coli O121 O-antigen comprises:

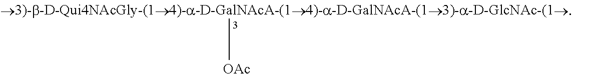

10. An immunogenic composition comprising the bioconjugate of any one of claims 1 to 9.

11. The immunogenic composition of claim 10 for use in treatment or prevention of an infection with Salmonella enterica.

12. The immunogenic composition of claim 10 for use in treatment or prevention of an infection with S. typhi.

13. A method of treatment or prevention of an infection with Salmonella enterica in a subject wherein the method comprises administering to the subject in need thereof an effective amount of the immunogenic composition of claim 10.

14. A method of treatment or prevention of an infection with S. typhi in a subject wherein the method comprises administering to the subject in need thereof an effective amount of the immunogenic composition of claim 10.

15. A prokaryotic host organism for generating a bioconjugate, wherein the pokaryotic host organism comprises:
a. a heterologous nucleotide sequence encoding a carrier protein comprising at least one glycosylation site comprising the amino acid sequence Asp/Glu-X-Asn-Z-Ser/Thr wherein X and Z may be any natural amino acid except Pro; and
b. a heterologous nucleotide sequence encoding an oligosaccaryltransferase;
wherein the prokaryotic host organism is recombinantly engineered to produce a modified E. coli O121 O-antigen and wherein the oligosaccharyl transferase transfers the modified E. coli O121 O-antigen to the Asn of the glycosylation site.

16. The prokaryotic host organism of claim 15, wherein the prokaryotic host organism is E. coli.

17. The prokaryotic host organism of claim 15, wherein the prokaryotic host organism is E. coli strain K12.

18. The prokaryotic host organism of claim 15, wherein the oligoshaccaryl transferase is PglB of Campylobacter jejuni.

19. A method of generating a bioconjugate of claim 1 wherein the method comprises:
a. culturing the prokaryotic host organism of any one of claims 15 to 18; and
b. isolating the bioconjugate.
